(19) United States
(12) Patent Application Publication
(10) Pub. No.: US 2024/0125728 A1
(43) Pub. Date: Apr. 18, 2024

(54) GENERALIZABLE NANOPORE SENSOR FOR HIGHLY SPECIFIC PROTEIN DETECTION AT SINGLE-MOLECULE PRECISION

(71) Applicants: Mohammad Ahmad, Syracuse, NY (US); Liviu Movileanu, Syracuse, NY (US)

(72) Inventors: Mohammad Ahmad, Syracuse, NY (US); Liviu Movileanu, Syracuse, NY (US)

(73) Assignee: SYRACUSE UNIVERSITY, Syracuse, NY (US)

(21) Appl. No.: 18/372,852

(22) Filed: Sep. 26, 2023

Related U.S. Application Data
(60) Provisional application No. 63/409,906, filed on Sep. 26, 2022.

(51) Int. Cl. G01N 27/327; G01N 33/543; G01N 33/68

(52) U.S. Cl. CPC G01N 27/3276; G01N 33/5438; G01N 33/6803

(57) ABSTRACT

A new class of sensing elements made of a protein pore fused to a programmable antibody-mimetic binder. This modular design expands the utility of nanopore sensors to numerous proteins while preserving their architecture, specificity, and sensitivity. The sensing elements were validated with protein analytes that drastically vary in size, charge, and structural complexity. These analytes produce unique electrical signatures that depend on their identity and quantity and the binder-analyte assembly at the nanopore tip.

### CROSS-REFERENCE TO RELATED APPLICATIONS

The present application claims priority to U.S. Provisional Application No. 63/409,906, filed on Sep. 26, 2022.

### STATEMENT REGARDING FEDERALLY SPONSORED RESEARCH AND DEVELOPMENT

This invention was made with government support under Grant GM088403 awarded by the National Institutes of Health (NIH). The government has certain rights in the invention.

### BACKGROUND OF THE INVENTION

### 1. Field of the Invention

The present invention relates to nanopore sensors and, more specifically, to a design that can be used to detect the specific protein analyte.

### 2. Description of the Related Art

Quantitative determination of protein biomarkers is a pressing necessity for disease prognostics, diagnostics, and therapeutics. Recent advancements in quantitative proteomics indicate that there are yet numerous unexplored proteins with potential implications for the progression of pathological conditions. Therefore, there is an increasing need for creating highly specific and sensitive protein sensing elements, which employ rapid signal responses to various biochemical stimuli. Molecular details of protein detection are illuminated using single-molecule technologies. In particular, single-molecule sensing with engineered nanopores using the resistive-pulse technique is adaptable to parallel recording technologies. Despite such a significant benefit, this approach usually requires that the targeted proteins be partitioned into the nanopore interior. Hence, the single-molecule observation is conducted under steric restrictions of the nanopore confinement, impairing the strength of native interactions and, in many cases, limiting its sensing specificity and sensitivity.

Detecting single proteins outside the nanopore is a practical alternative to sample the complexity of protein recognition events without the steric hindrance of the nanopore interior. This task would require a modular fusion protein in which an external protein binder (e.g., receptor) is covalently tethered to a nanopore through a flexible tether. However, a transducing mechanism is required to convert the physical captures and releases of a protein analyte (e.g., its ligand) into a specific electrical signature of the sensor. This challenge is exacerbated by the simple reason that protein binding events must occur away from the pore opening and outside the transmembrane electric field. In addition, changing the system to a new binder-analyte pair necessitates a lengthy and tedious optimization process that includes amplified difficulties. Earlier studies have suggested that these protein sensors may be limited to established protein fragments of ˜100 residues. For example, large protein binders likely induce additional steric constraints, precluding the clearance of the space around the pore opening. Moreover, the interaction interface of the binder must be fully accessible to the protein analyte. Therefore, the heterogeneous architecture, size, charge, and other traits of various protein binders suggest the need for an extensive effort in the protein engineering of an individual sensor for a given protein analyte. Hence, these sensing elements cannot be generalizable to many protein analytes.

### BRIEF SUMMARY OF THE INVENTION

The present invention is a new class of sensing elements for probing targeted proteins in solution at a single-detector precision. These sensors have an antibody-mimetic protein scaffold engineered on the tFhuA nanopore, a monomeric β-barrel scaffold. This design preserves the sensor's architecture and high sensitivity and specificity while featuring its generalization to numerous protein analytes. By changing only the binding interface, a novel binder-analyte pair can be obtained and readily implemented into these sensing elements. The binder is a monobody, a recombinant protein based on the 94-residue fibronectin type III domain (the FN3 scaffold). A monobody was fused to the N terminus of tFhuA, on the β-turns side, through a flexible (GGS)₂ tether. Using the monobody-based nanopore sensors with varying binding interfaces, it is possible to detect different proteins that vary substantially in charge, size, and structural complexity. When subjected to a challenging heterogeneous solution, this class of nanopore sensors can be used to identify and quantify a protein biomarker in a complex biofluid. Finally, direct measurements of time-resolved protein binding events at adjustable protein concentrations, without the steric hindrance of the nanopore interior, and within a substantially expanded dynamic range will influence nanoproteomics.

In one aspect, the present invention may be a sensor comprises of a lipid membrane having a predetermined membrane potential and a single polypeptide chain including a protein pore and an antibody-mimetic binder having a binding affinity for a target analyte tethered to the protein pore positioned in the membrane. The protein pore may comprise a monomeric β-barrel scaffold. The monomeric β-barrel scaffold may be a monomeric β-barrel scaffold of a tFhuA protein. The sensor may further comprise an FN3 monobody. The FN3 monobody may be tethered to the protein pore by a (GGS)₂ tether. The (GGS)₂ tether may be coupled to an N-terminus of the monomeric β-barrel scaffold of the tFhuA protein. The antibody-mimetic binder includes a tenth fibronectin type-III domain.

In another aspect, the present invention may be a method of detecting a target analyte. The method may include the step of providing a single polypeptide chain including a protein pore and an antibody-mimetic binder having a binding affinity for the target analyte. The method may further include the step of reconstituting the single polypeptide chain in a lipid membrane having a predetermined membrane potential to form a sensor. The method may additionally comprise the step of exposing the sensor to a solution that potentially contains the target analyte. The method further comprises measuring any change in the membrane potential. The protein pore may comprise a monomeric β-barrel scaffold. The monomeric β-barrel scaffold may be a monomeric β-barrel scaffold of a tFhuA protein. The sensor may further comprise an FN3 monobody. The FN3 monobody may be tethered to the protein pore by a (GGS)₂ tether. The (GGS)₂ tether may be coupled to an N-terminus of the monomeric β-barrel scaffold of the tFhuA protein. The antibody-mimetic binder includes a tenth fibronectin type-III domain.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of rational protein design of a versatile nanopore sensor for protein detection. (a) a tenth fibronectin type-III domain (FN3) or simply called monobody with FG, BC, and DE loops. (b) FN3's variants with binding loops in FN3 SUMO, Mb4, and Adnectin1, respectively. (c) tFhuA, a monomeric β-barrel scaffold. (d) Functional reconstitution of synthetic nanopore sensors into a planar lipid bilayer. The hSUMO1-binding monobody (FN3 SUMO) was fused to tFhuA via a flexible peptide tether. This single-polypeptide chain protein comprises a tFhuA protein pore, a flexible (GGS)₂ tether, and FN3SUMO (left). WDR5-binding monobody (Mb4) was fused to tFhuA, which was then reconstituted into a lipid bilayer (center). EGFR-binding monobody (Adnectin1) was fused to tFhuA in the same way as above (right). The protein-protein complexes of the monobodies with their cognate analytes are also shown. The structure of the three nanopore sensors were predicted by AlphaFold2.

FIG. 2 is an SDS-PAGE analysis of FN3SUMO-tFhuA, Mb4-tFhuA, and Adnectin1-tFhuA sensor proteins. The purity and size of all three proteins were checked by a 12% SDS-PAGE gel analysis. The expected molecular weights (MW) were observed on gel.

FIG. 3 is a structure prediction of FN3-tFhuA using AlphaFold2. (a) Amino acid sequence of FN3-tFhuA including the tether (SEQ ID NO: 1). (b) Predicted Local Distance Difference Test (pLDDT) per residue was between 80 and 100 for most residues. pLDDT per residue was extracted using AlphaFold2. (c) The structure of FN3-tFhuA predicted by AlphaFold2, demonstrating the orientation of the parent FN3 domain with respect to the central axis of tFhuA (red).

FIG. 4 is a structure prediction of FN3 SUMO-tFhuA, Mb4-tFhuA, and Adnectin1-tFhuA using AlphaFold2. (a) FN3SUMO-tFhuA. (b) Mb4-tFhuA. (c) Adnectin1-tFhuA. These structures were replicated in at least two independent run using AlphaFold2 on Google Colab server. Identical 3D structures were observed in each run.

FIG. 5 shows single-channel electrical traces of modified and unmodified tFhuA. (a) A representative single-channel electrical trace of FN3 SUMO-tFhuA, indicating a unitary conductance of ˜0.8 nS. This single-channel trace was low-pass Bessel filtered at a frequency of 3 kHz. (b) A representative single-channel electrical trace of Mb4-tFhuA, showing a unitary conductance of ˜1 nS. This single-channel electrical trace was low-pass Bessel filtered at a frequency of 3 kHz. (c) A representative single-channel electrical trace of Adnectin1-tFhuA, indicating a unitary conductance of ˜0.9 nS. This single-channel electrical trace was low-pass Bessel filtered at a frequency of 4 kHz. (d) A representative single-channel trace of the unmodified tFhuA, showing a unitary conductance of ˜1.5 nS. This single-channel trace was low-pass Bessel filtered at a frequency of 3 kHz. All traces were recorded at a transmembrane potential of +40 mV. All recordings were performed in 300 mM KCl, 10 mM Tris-HCl, pH 8.0. These recordings were replicated in n=3 independent experiments.

FIG. 6 shows normalized all-point histograms of the open-substate current. (a) FN3SUMO-tFhuA, (b) Mb4-tFhuA, (c) Adnectin1-tFhuA, and (d) tFhuA. All recordings were performed at an applied potential of +40 mV and in 300 mM KCl, 10 mM Tris-HCl, pH 8.0. These recordings were replicated in n=3 independent experiments.

FIG. 7 is the current-voltage curves of the three sensors and their comparisons with that of tFhuA. (a) FN3SUMO-tFhuA. (b) Mb4-tFhuA. (c) Adnectin 1-tFhuA. Here, I and ΔΨ indicate the current and applied transmembrane potential, respectively. Data are mean±s.d. from n=3 independent experiments.

FIG. 8 shows a series of graphs of real-time and label-free detection of hSUMO1. (a) Representative single-channel electrical traces of FN3SUMO-tFhuA in the presence of 0, 65, 130, and 260 nM hSUMO1. Oₒₙ and O_{off} are the hSUMO1-released and hSUMO1-captured substates, respectively. hSUMO1 was added to the cis compartment, which was at ground. These single-channel electrical signatures were replicated in n=3 independent experiments. The applied transmembrane potential was +40 mV. Single-channel electrical traces were further low-pass filtered at 3 kHz using an 8-pole Bessel filter. (b) A representative all-point current histogram of the Oₒₙ substate of FN3SUMO-tFhuA. The current amplitude (mean±s.e.m.) of the Oₒₙ substate was 32.1±0.1 pA. (c) A representative all-point current histogram of the Oₒₙ and O_{off} substates of FN3SUMO-tFhuA at 65 nM hSUMO1. The current amplitudes (mean±s.e.m.) of the Oₒₙ and O_{off} substates were 32.1±0.1 and 2.9±0.1 pA, respectively. (d) Semilogarithmic histograms of the hSUMO1-released durations (τₒₙ) at various hSUMO1 concentrations, [hSUMO1]. τₒₙ (mean±s.e.m.) were 125±4 ms (number of events: N=349), 69±5 ms (N=623), and 35±1 ms (N=1168) at [hSUMO1] values of 65 nM, 130 nM, and 260 nM, respectively. (e) Semilogarithmic histograms of the hSUMO1-captured durations (τ_{off}) at various [hSUMO1] values. τ_{off} (mean±s.e.m.) were 15±1 ms (N=354 events), 16±1 ms (N=633), and 14±1 ms (N=1180) at [hSUMO1] values of 65 nM, 130 nM, and 260 nM, respectively. (f) Plot illustrating the dependence of the event frequency in the form of 1/τₒₙ on [hSUMO1]. The slope of the linear fit of 1/τₒₙ versus [hSUMO1] is the association rate constant, kₒₙ, of FN3SUMO-hSUMO1 interactions, because kₒₙ=1/(τₒₙ[hSUMO1]). (g) Plot illustrating the dependence of 1/τ_{off} on [hSUMO1]. The horizontal line is an average fit of the (1/τ_{off}) data points recorded for various [hSUMO1] values. Data points in panels (f) and (g) represent mean±s.d. obtained from n=3 distinct experiments.

FIG. 9 is a representative raw trace of the FN3SUMO-tFhuA sensor when 65 nM hSUMO1 was added to the cis side of the chamber. (a) The single-channel trace was recorded in the absence of hSUMO1. (b) The single-channel trace was recorded in the presence of 65 nM hSUMO1. These traces were low-pass filtered using an 8-pole Bessel filter at a frequency of 7 kHz. The other experimental conditions are stated in Methods. This single-channel electrical signature was replicated in n=3 independent experiments.

FIG. 10 is a negative-control experiment for testing non-specific binding of hSUMO1 with the pore opening. (a) The unmodified tFhuA was functionally reconstituted into a lipid bilayer. (b) Upper panel is showing the signature when no hSUMO1 was added. tFhuA shows a conductance of ˜1.5 nS. In the lower panel, 200 nM hSUMO1 was added to the cis compartment. Very rare and brief current blockades were noted. The applied transmembrane potential was +40 mV. The buffer solution contained 300 mM KCl, 10 mM Tris-HCl, pH 8.0. Single-channel traces were low-pass filtered using an 8-pole Bessel at a frequency of 2 kHz. These recordings were replicated in n=3 independent experiments

FIG. 11 shows that the addition of hSUMO1 to the trans side did produce rare and brief current transitions when an FN3SUMO-tFhuA nanopore was functionally reconstituted into a lipid bilayer. (a) No hSUMO1 was added to the trans side. (b) 150 nM hSUMO1 was added to the trans side. This result confirmed the insertion of the FN3SUMO-tFhuA nanopore into a lipid bilayer with a preferred orientation. Single-channel electrical traces were low-pass Bessel filtered at a frequency of 4 kHz. These single-channel electrical traces were acquired in two replicates. All recordings were performed under an applied potential of +40 mV and in 300 mM KCl, 0.5 mM TCEP, 10 mM Tris-HCl, pH 8.0

FIG. 12 shows a series of graphs of the single-molecule sensing of WDR5. (a) Representative single-channel electrical traces of Mb4-tFhuA in the presence of 0, 50, 150, and 300 nM WDR5. Oₒₙ and O_{off} are the WDR5-released and WDR5-captured substates, respectively. These single-channel electrical signatures were replicated in n=3 independent experiments. The applied transmembrane potential was +40 mV. Single-channel electrical traces were further low-pass filtered at 3 kHz using an 8-pole Bessel filter. (b) A representative all-point current histogram of the Oₒₙ substate of Mb4-tFhuA. The current amplitude (mean±s.e.m.) of the Oₒₙ substate was 39.5±0.1 pA. (c) A representative all-point current histogram of the Oₒₙ and O_{off} substates of Mb4-tFhuA at 50 nM WDR5. The current amplitudes (mean±s.e.m.) of the Oₒₙ and O_{off} substates were 39.5±0.1 and 34.5±0.1 pA, respectively. (d) Semilogarithmic histograms of the WDR5-released durations (τₒₙ) at various WDR5 concentrations, [WDR5]. τₒₙ (mean±s.e.m.) were 178±6 ms (number of events: N=466), 65±7 ms (N=1175), and 34±4 ms (N=2235) at [WDR5] values of 50 nM, 150 nM and 300 nM, respectively. (e) Semilogarithmic histograms of the WDR5-captured durations (τ_{off}) at various [WDR5] values. τ_{off} (mean±s.e.m.) were 12±2 ms (N=441 events), 10±3 ms (N=1127), and 14±2 ms (N=2034) at [WDR5] values of 50 nM, 150 nM and 300 nM, respectively. (f) Plot illustrating the dependence of the event frequency in the form of 1/τₒₙ on [WDR5]. The slope of the linear fit of 1/τₒₙ versus [WDR5] is the association rate constant, kₒₙ, of Mb4-WDR5 interactions, because kₒₙ=1/(τₒₙ[WDR5]). (g) Plot illustrating the dependence of 1/τ_{off} on [WDR5]. The horizontal line is an average fit of the (1/τ_{off}) data points recorded for various [WDR5] values. Data points in panels (f) and (g) represent mean±s.d. obtained from n=3 distinct experiments.

FIG. 13 is a representative raw trace of the Mb4-tFhuA sensor when 150 nM WDR5 was added to the cis side of the chamber. (a) The single-channel trace was recorded in the absence of WDR5. (b) The single-channel trace was recorded in the presence of 150 nM WDR5 added to the cis side of the chamber. These traces were low-pass filtered using an 8-pole Bessel filter at a frequency of 7 kHz. The other experimental conditions are stated in Methods. This single-channel electrical signature was replicated in n=3 independent experiments.

FIG. 14 shows an experiment for testing non-specific binding of WDR5 with the pore opening (A) The unmodified tFhuA was assembled into lipid bilayer. (B) Fully open tFhuA shows a higher conductance (˜1.5 nS). The upper trace shows the signature when no WDR5 was added. In the lower panel, WDR5 was added to the cis compartment. Some non-specific binding events of WDR5 with tFhuA were noted in the form of low-amplitude and brief current spikes, likely due to collisions of WDR5 with the opening of tFhuA. The applied transmembrane potential was +40 mV. The buffer solution contained 300 mM KCl, 10 mM Tris-HCl, pH 8.0. These traces were replicated in n=3 independent experiments. Single-channel electrical traces were low-pass filtered using an 8-pole Bessel filter at a frequency of 2 kHz.

FIG. 15 shows that the addition of WDR5 to the trans side of a Mb4-tFhuA-containing lipid bilayer did not produce current transitions. (a) No WDR5 was added to the trans side. (b) 100 nM WDR5 was added to the trans side. This single-channel electrical signature was replicated in n=3 distinct experiments. This finding validated the successful insertion of the Mb4-tFhuA nanopore into the lipid bilayer in a favored orientation, as predicted by the previous studies. Single-channel electrical traces were low-pass filtered using an 8-pole Bessel filter at a frequency of 2 kHz. All recordings were performed under an applied potential of +40 mV, and in 300 mM KCl, 1 mM TCEP, 10 mM Tris-HCl, pH 8.0.

FIG. 16 is a schematic representation of biolayer interferometry (BLI) measurements. (a) In this case, Mb4-tFhuA_Cys287 was biotinylated at —SH₂ group of an engineered cysteine (C287) and immobilized onto streptavidin (SA) sensors surface. WDR5 was added to the wells. (b) Real-time optical measurements of WDR5-Mb4 interactions. 50 nM biotin-tagged Mb4-tFhuA_Cys287 was loaded onto the SA sensor surface. Titration series of WDR5 (1.5, 3.0 and 6.0 μM) were employed as protein analytes and the corresponding association and dissociation curves are shown for these WDR5 concentrations. These sensorgrams were acquired in n=6 replicates.

FIG. 17 is a representative raw trace of the Adnectin1-tFhuA sensor when 20 nM EGFR was added to the cis side of the chamber. (a) The single-channel trace was recorded in the absence of EGFR. (b) The single-channel trace was recorded in the presence of 20 nM EGFR added to the cis side of the chamber. These traces were low-pass filtered using an 8-pole Bessel filter at a frequency of 7 kHz. The other experimental conditions are stated in Methods. This single-channel electrical signature was replicated in n=3 independent experiments.

FIG. 18 shows an experiment for testing non-specific bindings of EGFR with the pore opening of tFhuA. (a) The unmodified tFhuA was functionally reconstituted into a lipid bilayer. (b) Fully open tFhuA shows a unitary conductance of ˜1.5 nS. In the upper trace, no EGFR was added. In the lower trace, 50 nM EGFR was added to the cis compartment. Some low-amplitude brief current spikes were noted, presumably due to non-specific binding events of EGFR to tFhuA. These events are perhaps due to high negative charge of EGFR at pH 8.0 (Table 1). The applied transmembrane potential was +20 mV. The buffer solution contained 300 mM KCl, 10 mM Tris-HCl, pH 8.0. These traces were replicated in n=3 independent experiments. Single-channel electrical traces were low-pass filtered using an 8-pole Bessel filter at a frequency of 2 kHz.

FIG. 19 is a series of graphs of EGFR exhibiting a bimodal protein recognition with Adnectin1. (a) Representative single-channel electrical traces of Adnectin1-tFhuA in the presence of 0, 10, 20, and 40 nM EGFR. Oₒₙ and O^{off} are the EGFR-released and EGFR-captured substates, respectively. These single-channel electrical signatures were replicated in n=3 independent experiments. The applied transmembrane potential was +20 mV. Single-channel electrical traces were further low-pass filtered at 2 kHz using an 8-pole Bessel filter. (b) Event histograms of the normalized current blockades A/I₀, where A and I₀ are the current amplitude of individual blockades and the current amplitude of the Oₒₙ substate, respectively. The cumulative fits are marked in green. The blue and black curves indicate fits of low- and large-amplitude current blockades, respectively. For 10 nM EGFR, these values (mean±s.e.m.) were (65.0±0.3)% and (84.5±0.3)%, respectively (number of events, N=467). For 20 nM EGFR, they were (65.2±0.2)% and (85.1±0.2)%, respectively (N=924). For 40 nM EGFR, they were (65.3±0.2)% and (85.5±0.2)%, respectively (N=1711). (c) Semilogarithmic histograms of the EGFR-released durations (τₒₙ) at various EGFR concentrations, [EGFR]. τₒₙ (mean±s.e.m.) were 0.78±0.04 s (number of events: N=491), 0.41±0.03 s (N=843), and 0.25±0.02 s (N=1641) at [EGFR] values of 10 nM, 20 nM and 40 nM, respectively. (d) Semilogarithmic EGFR-captured durations (τ_{off}) sing at various [EGFR] concentrations. The cumulative fits are marked in black. The red and cyan curves indicate fits for short- and long-lived EGFR captures, respectively. For 10 nM EGFR, they (mean±s.e.m.) were 0.072±0.011 s and 1.2±0.1 s, respectively (number of events: N=441). For 20 nM EGFR, they were 0.069±0.007 s and 0.96±0.09 s, respectively (N=806). For 40 nM EGFR, they were 0.066±0.006 s and 0.81±0.11 s, respectively (N=1598). (e) Plot illustrating the dependence of the event frequencies in the form of 1/τₒₙ₋ᵢ on [EGFR]. τₒₙ₋₁ and τₒₙ₋₂ are the EGFR-released durations between the short- and long-lived EGFR captures, respectively. The slopes of the linear fits of 1/τₒₙ₋ᵢ versus [EGFR] are the association rate constant, kₒₙ₋ᵢ, of Adnectin1-EGFR interactions, because kₒₙ₋ᵢ=1/(τₒₙ₋ᵢ[EGFR]). (f) Plot illustrating the dependence of 1/τ_{off-i} on [EGFR]. The red and green horizontal lines are average fits of the (1/τ_{off-1}) and (1/τ_{off-2}) data points recorded for various [EGFR] values, respectively. Here, i=1 and i=2 stand for subscripts corresponding to the short- and long-lived EGFR captures, respectively. Data points in panels (e) and (f) represent mean±s.d. obtained from n=3 distinct experiments.

FIG. 20 shows the structures of Adnectin1 and EGF in complex with EGFR. (a) Specific binding between domain I of EGFR and Adnectin1. Adnectin1 is in blue (cartoons), and EGFR surface is represented by various colors according to different domains (3QWQ.pdb). (b) Interaction between EGF and domain I of EGFR. EGF is shown in magenta (cartoons) and EGFR as surface by various colors (1NQL.pdb). (c) Overlap of Adnectin1 and EGF contacting surfaces on EGFR domain I is shown. Adnectin 1 (blue) and EGF (magenta) are represented as cartoons but domain I is represented as surface (red). (d) Adnectin1 and EGF showing the hydrogen bonds (yellow) at domain I. The unique overlapping and contacting surfaces are shown as Adnectin 1(blue), EGF (magenta), and EGFR domain I (red).

FIG. 21 is the interconversion-independent and interconversion-dependent kinetic model. (a) The interconversion-independent kinetic model. This model assumes no transitions between different EGFR-captured substates, C₁ and C₂. The schematic of the model. 0 represents the EGFR-released substate. C₁ and C₂ indicate the short- and long-lived EGFR-captured substates, respectively. Q-matrix of the interconversion-independent three-substate kinetic model is also presented in the same panel. (b) The interconversion-dependent kinetic model. This model assumes transitions between the EGFR-captured substates C₁ and C₂. O represents the EGFR-released substate. C₁ and C₂ indicate the short- and long-lived EGFR-captured substates, respectively. The interconversion rate constants show transitions between different substates. Here, the first digit denotes the initial state, and the second digit indicates the final state. Q-matrix of the interconversion-independent three-substate kinetic model is also presented in the same panel. Transitions from the EGFR-captured substates to EGFR-released substates and vice-versa are marked by magenta arrows. Transitions between the EGFR-captured substates are marked in green.

FIG. 22 is a positive-control experiment for testing the specificity of Adnectin1-tFhuA sensor in the presence of hSUMO1. These are single-channel electrical recordings with Adnectin1-tFhuA when hSUMO1 was added to the cis chamber. The applied potential was +20 mV. (a) 0 nM hSUMO1. (b) 150 nM hSUMO1. (c) 450 nM hSUMO1. All recordings were performed in 300 mM KCl, 0.5 mM TCEP, 10 mM Tris-HCl, pH 8.0. The single-channel electrical traces were low-pass filtered at 2 kHz using an 8-pole Bessel filter. Specific hSUMO-captured events were not observed with the Adnectin1-tFhuA sensor. These single-channel electrical signatures were replicated in n=4 independent experiments

FIG. 23 is a positive-control experiment for testing the specificity of Adnectin1-tFhuA sensor in the presence of WDR5. These are single-channel electrical recordings with Adnectin1-tFhuA when WDR5 was added to the cis chamber. The applied potential was +20 mV. (a) 0 nM WDR5. (b) 200 nM WDR5. (c) 600 nM hSUMO1. All recordings were performed in 300 mM KCl, 1 mM TCEP, 10 mM Tris-HCl, pH 8.0. The single-channel electrical traces were low-pass filtered at 2 kHz using an 8-pole Bessel filter. Specific WDR5-captured events were not observed with the Adnectin1-tFhuA sensor. These single-channel electrical signatures were replicated in n=3 independent experiments.

FIG. 24 shows a series of graphs of single-molecule detection and quantification of EGFR in a heterogenous solution. (a) A representative single-channel electrical trace of Adnectin1-tFhuA. (b) The trace in (a) in the presence of 20 nM EGFR. (c) The trace in (b) in the presence of 5% (v/v) FBS. The transmembrane potential was +20 mV. This subset of single-channel electrical signatures was replicated in n=3 independent experiments. Single-channel electrical traces were further low-pass filtered at 500 Hz using an 8-pole Bessel filter. (d) Power spectral density (PSD) of current noise of traces illustrated in panels (a)-(c). Each spectrum represents an average of three independent traces. (e) A representative all-point current histogram of the Om substate of Adnectin1-tFhuA. The current amplitude (mean±s.e.m.) of the Oₒₙ substate was 18.2±0.1 pA. (f) A representative all-point current histogram of the Oₒₙ and O_{off} substates of Adnectin1-tFhuA at 20 nM EGFR. The current amplitudes (mean±s.e.m.) of the O_{off-1} and O_{off-2} substates were 6.40±0.01 pA, and 2.34±0.01 pA, respectively. (g) A representative all-point current histogram of the Oₒₙ and O_{off} substates of Adnectin1-tFhuA at 20 nM EGFR and in the presence of 5% fetal bovine serum (FBS). This plot reveals the residual signal produced by the FBS constituents (I_{FBS}). (h) A semilogarithmic histogram of the EGFR-released durations (τₒₙ) at 20 nM EGFR. τₒₙ (mean±s.e.m.) was 0.396±0.030 s (number of events: N=844). (i) A semilogarithmic histogram of the EGFR-captured durations (τ_{off}) at 20 nM EGFR. τ_{off-1} and τ_{off-2} (mean±s.e.m.) were 0.044±0.015 s and 0.982±0.049 s, respectively (number of events: N=734). (j) A semilogarithmic histogram of the EGFR-released durations (τₒₙ) at 20 nM EGFR and in the presence 5% FBS. τₒₙ (mean±s.e.m.) was 0.607±0.051 s (n=738). (k) A semilogarithmic histogram of the EGFR-captured durations (τ_{off}) at 20 nM EGFR and in the presence 5% FBS. τ_{off-1} and τ_{off-2} (mean±s.e.m.) were 0.036±0.013 s and 0.806±0.078 s, respectively (N=694). In panels (i) and (k), the cumulative fits are marked in black. The blue and green curves indicate fits for short- and long-lived captures, respectively.

FIG. 25 shows side and top views of the modelled structure of the hSUMO1-FN3SUMO-tFhuA complex. (a) FN3SUMO is oriented approximately 90° with respect to the central axis of tFhuA (side view), as judged by the AlphaFold2 approach. Binding of hSUMO1 to FN3SUMO almost fully blocks the pore opening (top view). (b) This panel shows the functionally reconstituted FN3SUMO-tFhuA into a lipid bilayer in the hSUMO1-released (Oₒₙ) and hSUMO1-captured (O_{off}) substates.

FIG. 26 shows side and top views of the modelled structure of the WDR5-Mb4-tFhuA complex. (a) Mb4 is oriented approximately 90° with respect to the central axis of tFhuA (side view), as judged by the AlphaFold2 approach. Binding of WDR5 to Mb4 partly blocks the pore opening (top view). (b) This panel shows the functionally reconstituted Mb4-tFhuA into a lipid bilayer in the WDR5-released (Oₒₙ) and WDR5-captured (O_{off}) substates.

FIG. 27 are side and top view of the modelled structure of the EGFR-Adnectin1-tFhuA complex. (a) The Adnectin1 is oriented approximately 90° with respect to the central axis of tFhuA (side view), as judged by the AlphaFold2 approach. Binding of EGFR to Adnectin1 almost fully blocks the pore opening (top view). (b) This panel shows the functionally reconstituted Adnectin1-tFhuA into a lipid bilayer in the EGFR-released (Oₒₙ) and EGFR-captured (O_{off}) substates.

FIG. 28 is a schematic of the relative current blockades acquired with FN3SUMO-tFhuA, Mb4-tFhuA, and Adnectin1-tFhuA. (a) Graphic representation of stochastic sensing of hSUMO1 using an FN3SUMO-tFhuA sensor, which maintains a basal open-state current (left panel). When hSUMO1 is added to the cis side, the analyte is expected to produce current transitions between two current substates (right panel). (b) Mb4-tFhuA sensor maintains a basal open-state current (left panel). When added to the cis side, WDR5 is expected to produce current transitions (right panel). (c) The Adnectin1-tFhuA protein maintains a basal open-state current (left panel). When added to the cis side, EGFR is expected to produce current transitions (right panel)

FIG. 29 are linear fits of the analyte-captured durations acquired with FN3SUMO-tFhuA, Mb4-tFhuA, and Adnectin1-tFhuA sensors. (a) hSUMO1. (b) WDR5. (c) EGFR. The applied transmembrane potentials were +40 mV, +40 mV, and +20 mV, respectively. All the other experimental conditions are the same as those mentioned in Methods. Plot values indicate mean±s.d. from n=3 independent experiments.

FIG. 30 shows steady-state FP anisotropy curves of FN3SUMO-tFhuA-hSUMO1 and Mb4-tFhuA-WDR5 interactions. (a) hSUMO1 was labelled with fluorescein. The final concentration of the labeled hSUMO1 in each well was 50 nM. (b) WDR5 was labelled with rhodamine. The final concentration of the labeled WDR5 in each well was 50 nM. The labelled hSUMO1 and WDR5 were titrated against FN3SUMO-tFhuA and Mb4-tFhuA, respectively. Data indicate mean±s.d. from n=3 independent experiments.

FIG. 31 is an SDS-PAGE gel of purified EGFR. The EGFR was purified using a polyhistidine-tag column. The protein purity was checked on a 4-20%-gradient SDS-PAGE gel. The expected molecular weight (MW) of EGFR is 69.6 kDa. However, we observed a higher apparent MW due to glycosylation.

FIG. 32 is a schematic of a rational protein design of a versatile nanopore sensor for protein detection that uses an affibody.

### DETAILED DESCRIPTION OF THE INVENTION

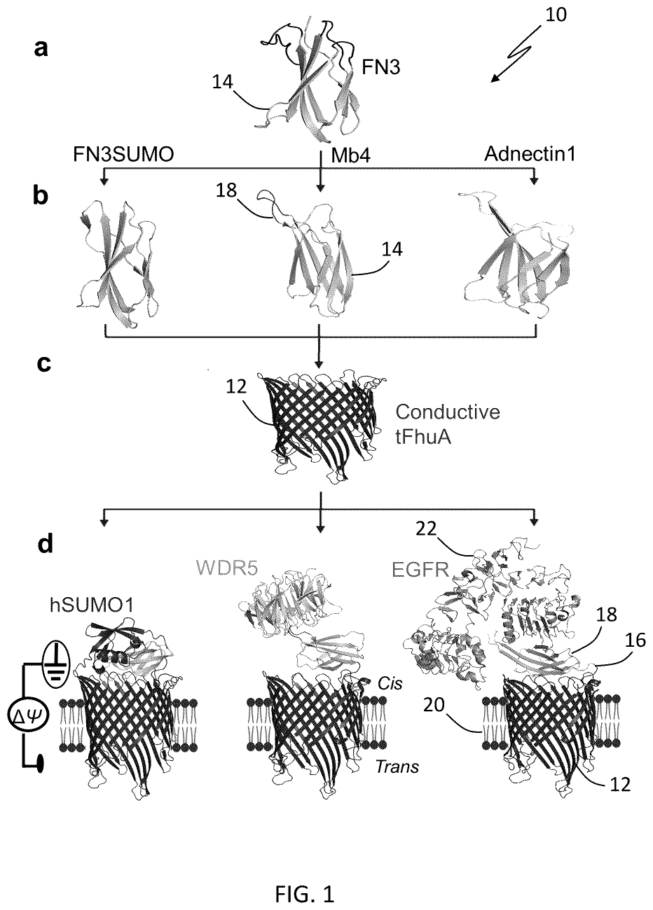

Referring to the figures, wherein like numerals refer to like parts throughout, there is seen in FIG. 1 a schematic of a monobody-based nanopore sensor 10 according to the present invention. Sensor 10 comprises a protein pore 12, a monobody scaffold 14 coupled to the pore by a tether 16, and an antibody mimetic binding site 18 that is specific to whichever target analyte is to be detected supported by monobody scaffold 14. As seen in FIG. 1, the combined pore 12, scaffold 14, tether 16 and binding site 18 are incorporated into a membrane 20 to provide sensor 10. Sensor 10 has a measurable membrane potential prior to binding site 18 binding to the target analyte that will change when binding occurs so that the resulting change in membrane potential indicates that the target analyte has indeed bound to the binding site 18. Sensor 10 is particularly effective in detecting the presence of an analyte in extremely low concentrations in complex environments such as blood and other fluid samples. Scaffold 14 is preferably a protein of less than 150 residues and serves to stabilize the particular binding site 18 selected for detecting a target analyte 22. In various examples below, a fibronectin type III domain (FN3) was used as scaffold 14 to support and stabilize binding sites 18 targeting specific analytes 22 as follows: (i) human small ubiquitin-related modifier (hSUMO1), a model protein with implications in various cellular processes, such as DNA damage repair, chromosome dynamics, and cell cycle; (ii) WD40 repeat protein 5 (WDR5), a chromatin-associated protein hub known for its involvement in the epigenetic regulation of histone 3 lysine 4 (H3K4) methylation; (iii) epidermal growth factor receptor (EGFR), a prognosis protein biomarker in lung, colorectal, and breast cancers. In other example, an affibody was used to support a binding site 18 for human epidermal growth factor 2 (HER2) as target analyte 22. It should be recognized by those of skill in the art that other scaffolds of less than 150 residues may be used to stabilize the particular binding site chose for the target analyte, and that any known protein binding sites may be incorporated into the scaffold to detect the target analyte of interest. In the example of FN3, the existing binding sites (shown in the top of FIG. 1) were replaced with the appropriate binding sites known in the field for the specific target analytes, i.e., the binding sites for hSUMO1, WDR5, and EGFR.

### EXAMPLE 1

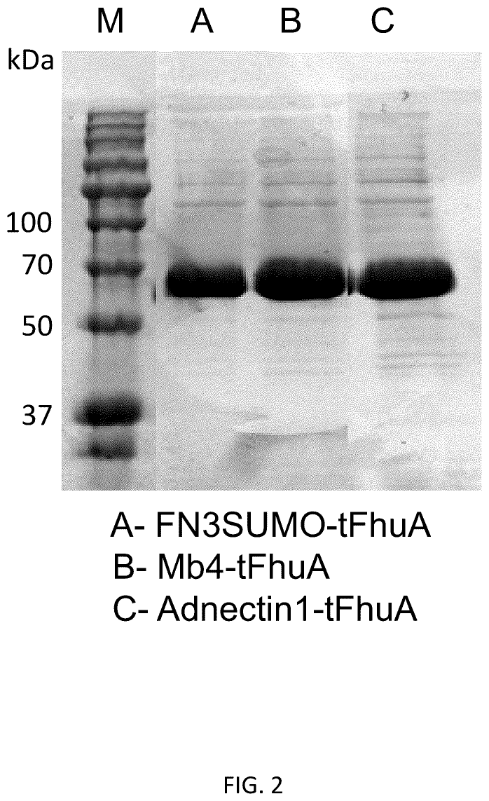

As explained above, the present invention was implemented by creating three sensors 10 using FN3SUMO, Mb4, and Adnectin1 monobodies as binders against hSUMO1, WDR5, and the ectodomain of EGFR, respectively. These monobody-based sensors are denoted by FN3SUMO-tFhuA, Mb4-tFhuA, and Adnectin1-tFhuA, respectively (FIG. 2).

TABLE 1
Comparison of the size, charge, and structural
complexity of three protein analytes.

| | Size | | Structural |
|---|---|---|---|
| Protein analytes | (kDa) | Charge* | organization |
| hSUMO1 | 11.1 | −5.8 | 3 α helices |
| | | | and 4 β strands |
| WDR5 | 36.5 | 1.9 | 7-bladded beta- |
| | | | propeller fold with |
| | | | a total of 28 β |
| | | | strands |
| EGFR | 69.2 | −16.3 | Four distinct |
| | | | structural domains |

*Charges of these protein analytes were calculated using protein calculator v3.4 by amino acid sequence at pH 8.0.

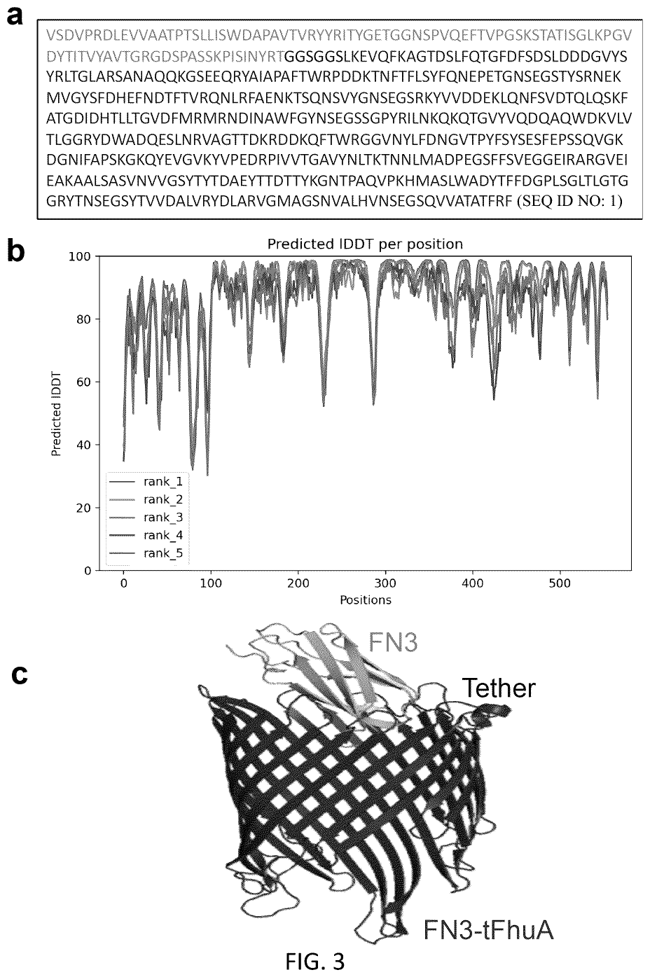

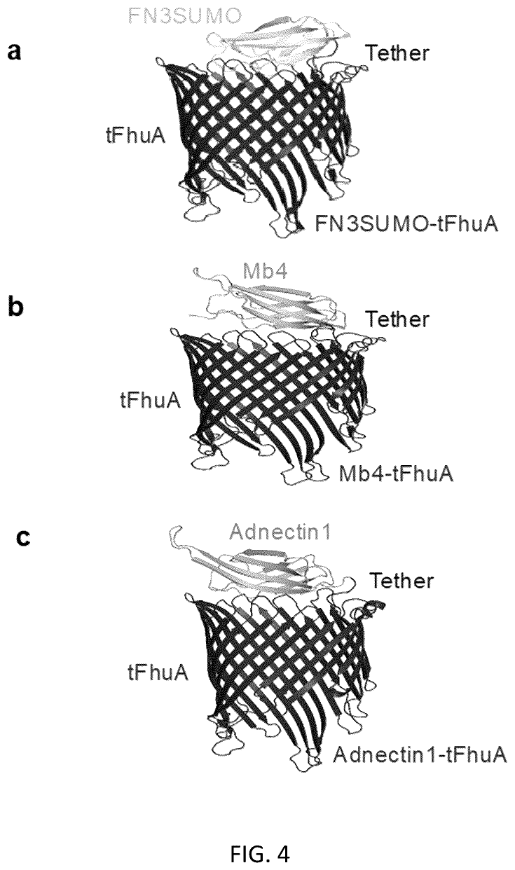

An artificial intelligence approach was used to elucidate the overall three-dimensional conformation of a folded protein using its amino acid sequence. The most suited structural model for FN3-tFhuA was reached when predicted Local Distance Difference Test (pLDDT), a confidence score per residue, was between 80 and 100 for most residues (FIG. 3ab). Remarkably, this model illustrates that FN3 orients almost 90° with respect to the central axis of tFhuA (FIG. 3c). Similar results were obtained with FN3SUMO-tFhuA, Mb4-tFhuA, and Adnectin1-tFhuA (FIG. 4). Therefore, FN3 monobodies in all sensors potentially block a significant ionic flow of the tFhuA pore due to their orientation with respect to the central axis of tFhuA.

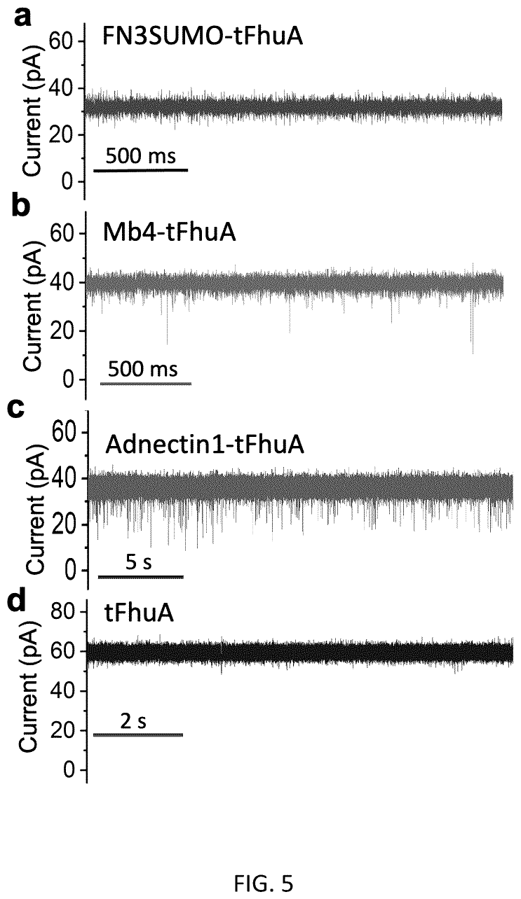

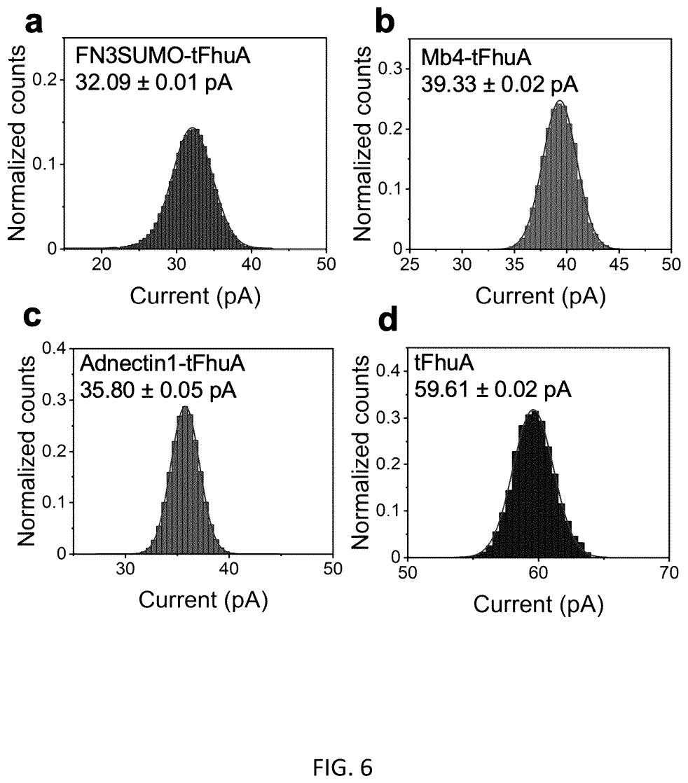

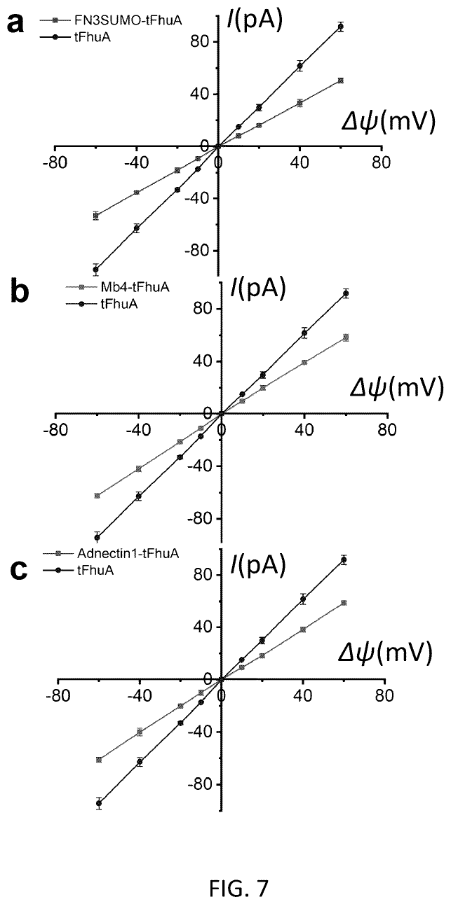

An inspection of all sensors at a transmembrane potential of +40 mV revealed a relatively quiet single-channel electrical current recorded with FN3 SUMO-tFhuA and Mb4-tFhuA, and a slightly noisy signal acquired with Adnectin1-tFhuA (FIGS. 5 and 6). The unitary conductance of FN3 SUMO-tFhuA, Mb4-tFhuA, and Adnectin1-tFhuA were (mean±s.d.) 0.81±0.03 nS, 0.99±0.04 nS, and 0.90±0.02 nS (Table 2), respectively. These are significant reductions in the unitary conductance compared that value of the unmodified tFhuA (1.52±0.10 nS) (FIG. 7). This finding is in accordance with the predictions made by the AlphaFold2 approach.

TABLE 2
Unitary conductance values of the engineered monobody-based sensors,
and their comparison with the unmodified tFhuA nanopore.

| Nanopore sensor | Applied potential (mV) | Conductance (nS) |
|---|---|---|
| FN3SUMO-tFhuA | +40 | 0.81 ± 0.03 |
| Mb4-tFhuA | +40 | 0.99 ± 0.04 |
| Adnectin1-tFhuA | +40 | 0.90 ± 0.02 |
| tFhuA (control) | +40 | 1.52 ± 0.10 |

Values represent mean±s.d. from n=3 distinct experiments.

Real-Time Protein Detection Using FN3SUMO-tFhuA

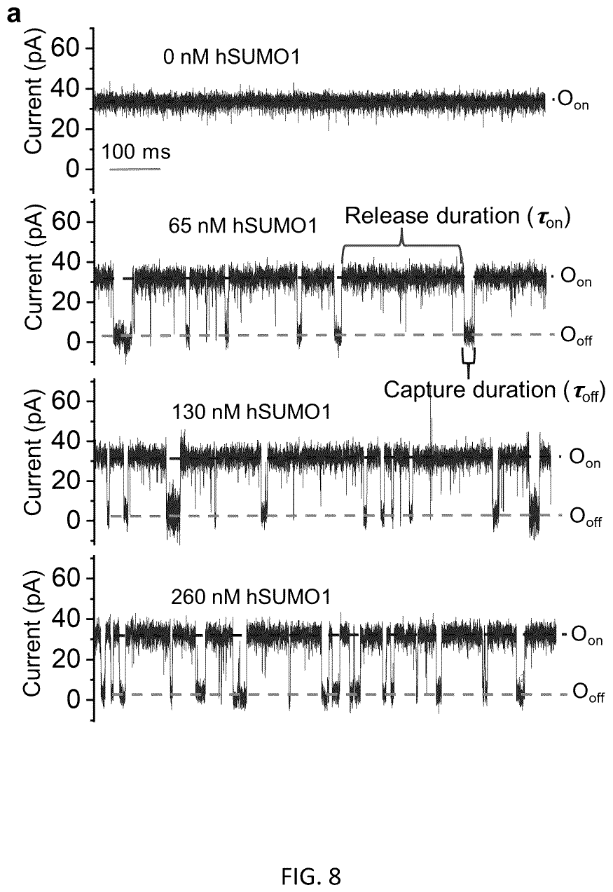

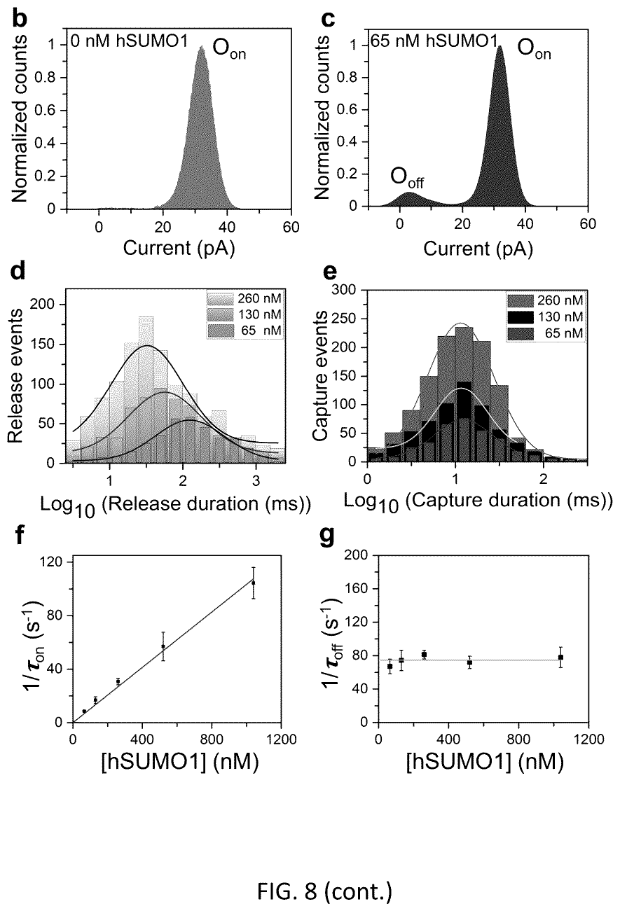

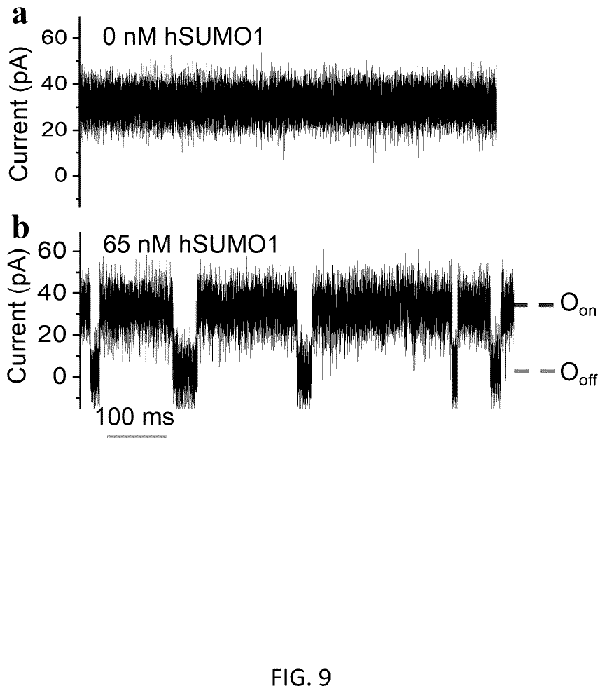

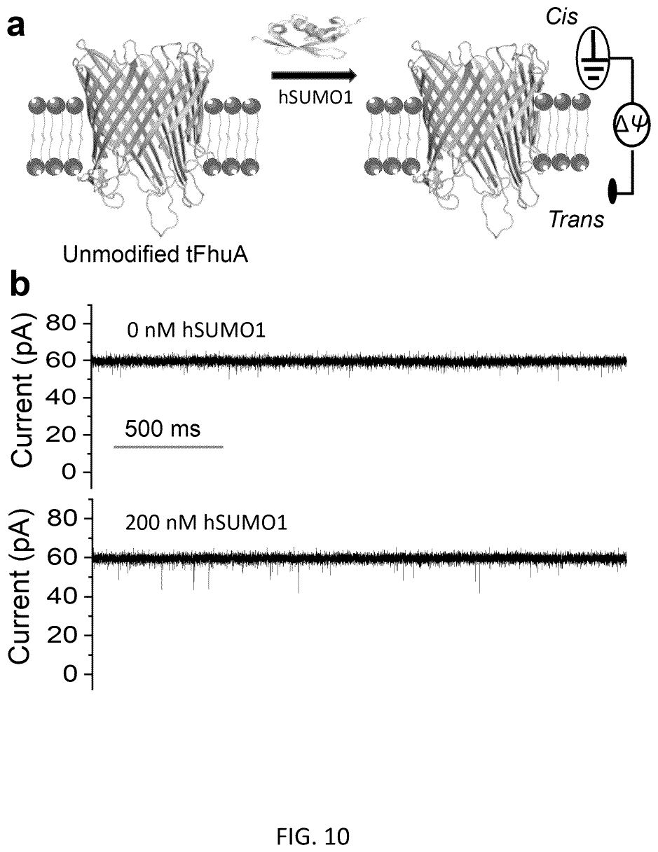

FN3SUMO-tFhuA was first functionally reconstituted into a membrane at an applied transmembrane potential of +40 mV. The presence of hSUMO1 in the cis compartment at nanomolar concentrations produced frequent current blockades (FIG. 8a; FIG. 9) between Oₒₙ open substate and O_{off} closed substate. Their normalized current amplitude, A/I₀, was (91.5±0.7)%, where A and I₀ are the current amplitude of the blockades and the open-state current, respectively (FIG. 8ab). Yet, infrequent and brief current spikes were observed when hSUMO1 was added to the cis side of a tFhuA sensor-containing bilayer (FIG. 10). Taken together, these control measurements indicate that hSUMO1 did not produce any significant current blockades due to nonspecific interactions with the cis opening of the nanopore.

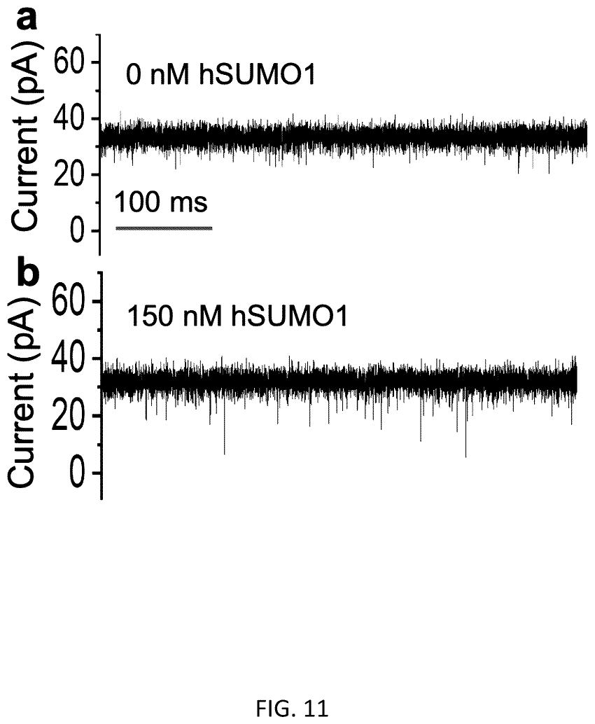

Moreover, hSUMO1-captured events were noted in a concentration-dependent manner when hSUMO1 was added to the cis compartment (FIG. 8). hSUMO1-released and hSUMO1-captured events recorded with FN3SUMO-tFhuA corresponded to the open-substate, Oₒₙ, and closed-substate, O_{off}, respectively. However, hSUMO-captured events were not detectable when hSUMO1 was added to the trans compartment (FIG. 11). This finding agrees with the previous studies that tFhuA and its derivatives insert into the membrane with a single orientation.

Next, detailed statistical analyses were performed of both the hSUMO1-released and hSUMO1-captured durations, whose mean values were denoted by τₒₙ and τ_{off}, respectively. The maximum likelihood method and logarithm likelihood ratio (LLR) tests were employed to determine the most accurate distribution model of these time constants. Durations of hSUMO1-released and hSUMO1-captured events showed a single-exponential distribution in the form of a single peak in a semilogarithmic representation (FIG. 8de). Note that the center location of the peak is the logarithm of the time constant. Increasing the hSUMO1 concentration, [hSUMO1], decreased the τₒₙ but did not alter τ_{off} (Table 3).

TABLE 3
Mean values of the release (τₒₙ) and capture durations (τ_{off})
of hSUMO1-FN3SUMO interactions using an FN3SUMO-tFhuA sensor.

| [hSUMO1] (nM) | τₒₙ (ms) | τ_{off} (ms) |
|---|---|---|
| 65 | 116 ± 8 | 15 ± 2 |
| 130 | 60 ± 9 | 13 ± 3 |
| 260 | 32 ± 3 | 12 ± 1 |
| 520 | 18 ± 4 | 14 ± 3 |
| 1040 | 10 ± 1 | 13 ± 2 |

Values are mean±s.d. from n=3 independent experiments. The applied transmembrane potential was +40 mV. The buffer solution contained 300 mM KCl, 10 mM Tris-HCl, 0.5 mM TCEP, pH 8.0.

Here, the association rate constants, kₒₙ, were consistent for all [hSUMO1] values (Table 4). In addition, the frequency of hSUMO1-captured events, f, where f=1/τₒₙ, was proportional to [hSUMO1] in a ratio 1:1 (FIG. 8f), indicating a bimolecular association process of the hSUMO1-FN3SUMO complex. Using the linear fit of f([hSUMO1]), we obtain a kₒₙ value (mean±s.e.m.) of (1.12±0.02)×10⁸ M⁻¹s⁻¹. Dissociation rate constant k_{off} was determined as reciprocal of the mean hSUMO1-captured durations (1/τ_{off}). This value was independent of [hSUMO1] (FIG. 8g; Table 4), indicating a unimolecular dissociation mechanism of the hSUMO1-FN3SUMO complex. A linear fit of k_{off}([hSUMO1]) versus [hSUMO1] resulted in its mean±s.e.m. value of 74.5±2.4 s⁻¹, to yield a K_{D} of 665±24 nM (Table 5).

TABLE 4
Mean values of the association (kₒₙ) and dissociation
(k_{off}) rate constants of hSUMO1-FN3SUMO interactions.

| [hSUMO1] (nM) | kₒₙ (M⁻¹s⁻¹) × 10⁻⁸ | k_{off} (s⁻¹) |
|---|---|---|
| 65 | 1.3 ± 0.1 | 67 ± 9 |
| 130 | 1.3 ± 0.2 | 74 ± 12 |
| 260 | 1.2 ± 0.1 | 81 ± 5 |
| 520 | 1.1 ± 0.2 | 72 ± 7 |
| 1040 | 1.0 ± 0.1 | 78 ± 12 |

Values are mean±s.d. from n=3 independent experiments. The applied transmembrane potential was +40 mV. The buffer solution contained 300 mM KCl, 10 mM Tris-HCl, 0.5 mM TCEP, pH 8.0.

TABLE 5
The association (kₒₙ) and dissociation (k_{off}) rate constants
and equilibrium dissociation constant (K_{D}) of hSUMO1-FN3SUMO
interactions. kₒₙ value is the slope of the linear fit in FIG. 2f. k_{off}
value is the axis intercept of the horizontal line fit in FIG. 2g. Here, K_{D}
was calculated using the equation K_{D} = k_{off}/kₒₙ.

| kₒₙ (M⁻¹s⁻¹) × 10⁻⁸ | k_{off} (s⁻¹) | K_{D} (nM) |
|---|---|---|
| 1.12 ± 0.02 | 74.5 ± 2.4 | 665 ± 24 |

Values are provided as mean±s.e.m. The other experimental conditions were the same as those stated in Methods.

Detection of a Chromatin-Associated Protein Hub Using Mb4-tFhuA

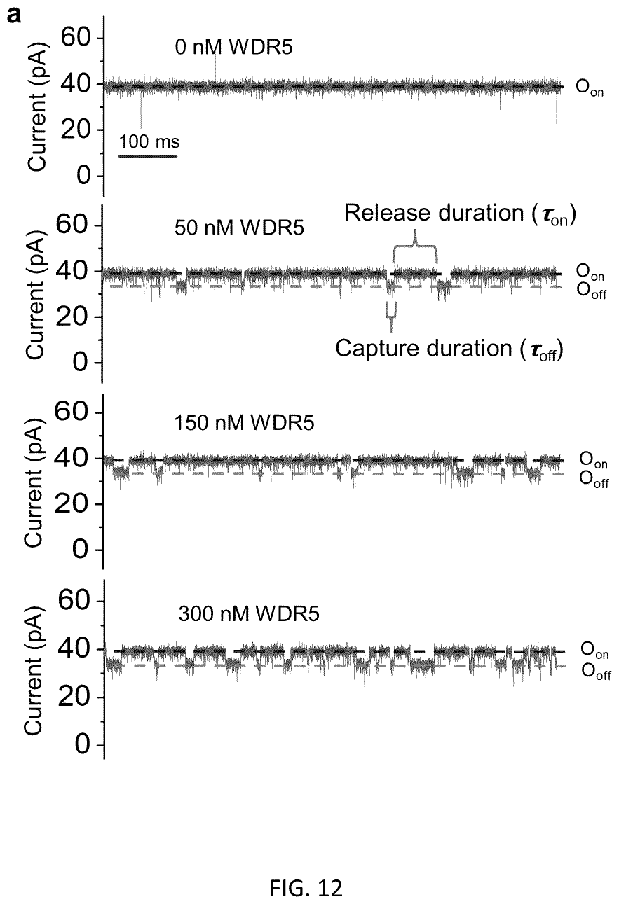

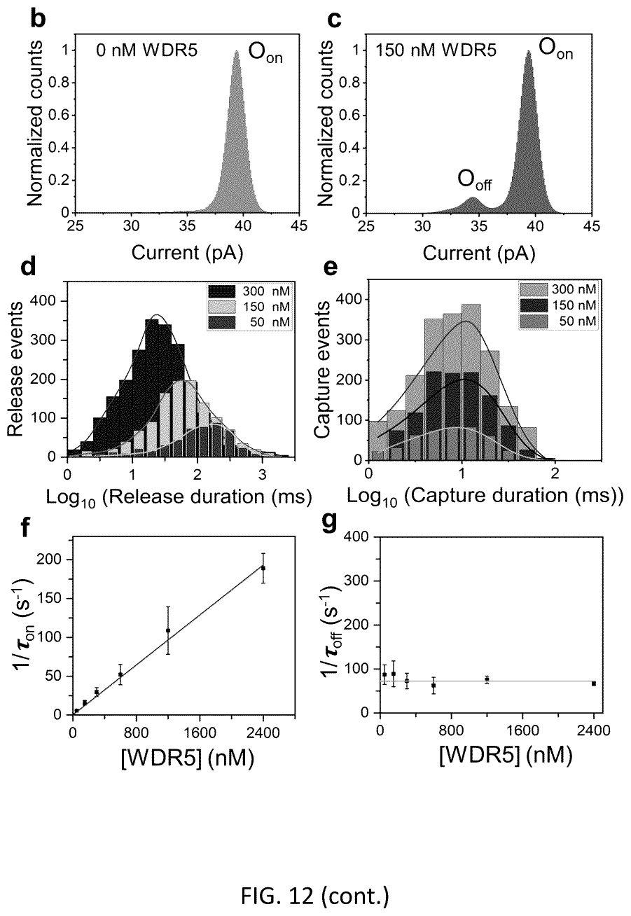

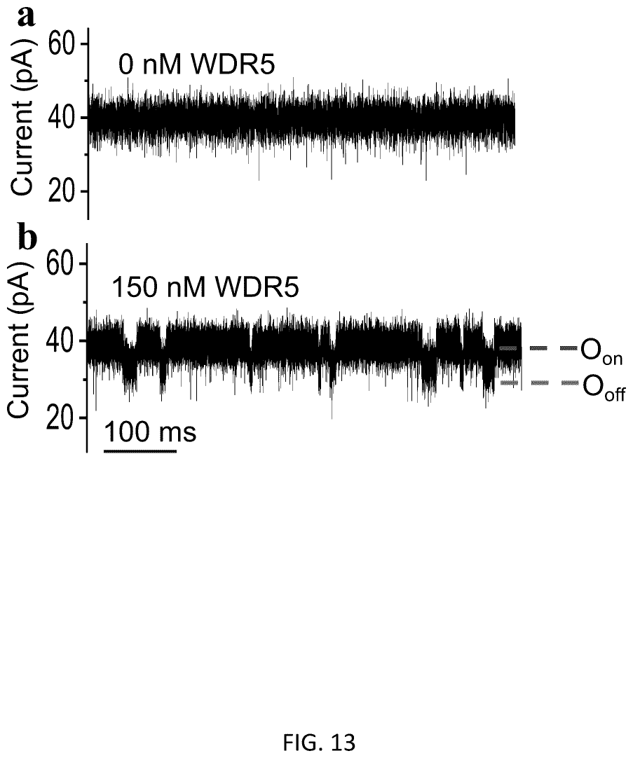

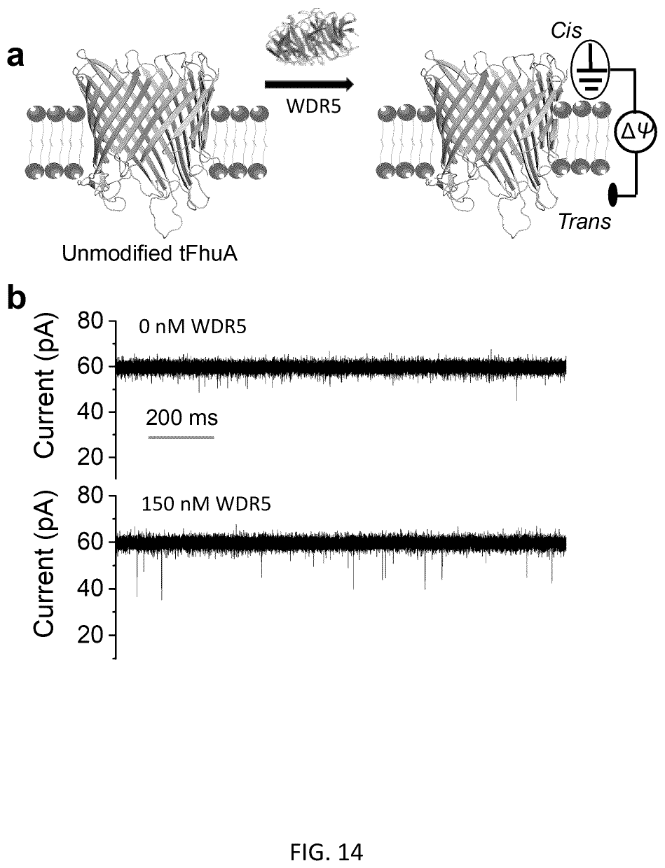

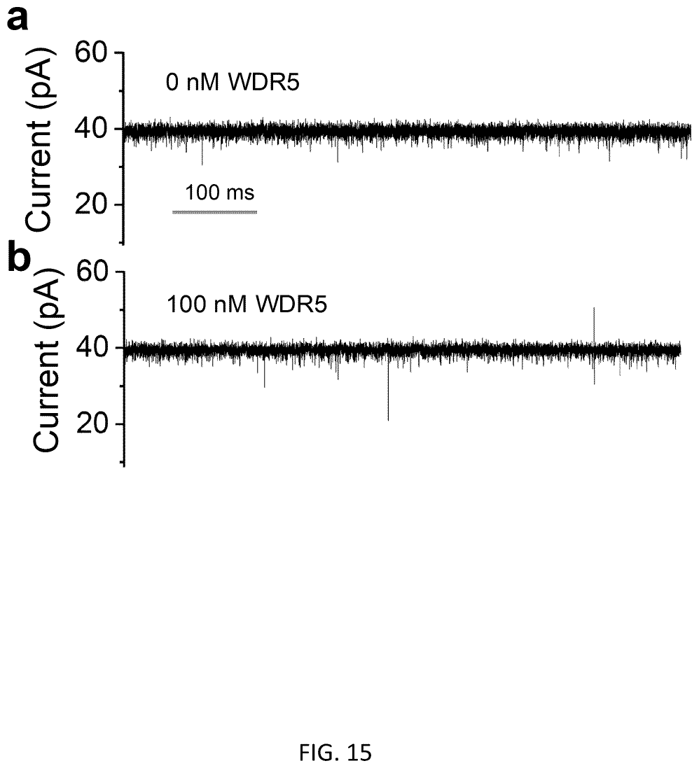

The same approach was used to detect WDR5 using a functionally reconstituted Mb4-tFhuA sensor into a lipid bilayer. When added to the cis compartment at nanomolar concentrations, WDR5 produced frequent current blockades (FIG. 12a; FIG. 13) between Oₒₙ open substate and O_{off} partly closed substate with a normalized current amplitude (14±1)% (FIG. 12bc). Again, this kind of current blockades was not noted when an unmodified tFhuA was exposed to WDR5 added to the cis side (FIG. 14) or when Mb4-tFhuA was exposed to WDR5 added to the trans side (FIG. 15). These findings suggest that WDR5-induced current blockades are brought about by specific WDR5-Mb4 interactions. WDR5-released (Oₒₙ) and WDR5-captured (O_{off}) events also followed a single-exponential distribution (FIG. 12de). In addition, the frequency of WDR5-captured events was proportional to its concentration, [WDR5] (FIG. 12f), whereas their duration was independent of [WDR5] (FIG. 12e, FIG. 12g; Tables 6-7). Using linear fits of the functions f([WDR5]) and k_{off}([WDR5]), we obtained a kₒₙ value (mean±s.e.m.) of (0.83±0.01)×10⁸ M⁻¹s⁻¹ and a k_{off} value (mean±s.e.m.) of 72.4±3.7 s⁻¹, to yield a K_{D} of 872±45 nM (Table 8). It should be noted the kinetics of WDR5-Mb4 interactions undergo fast association and dissociation rate constants.

TABLE 6
Mean values of the release (τₒₙ) and capture durations (τ_{off})
of WDR5-Mb4 interactions using an Mb4-tFhuA sensor.

| [WDR5] (nM) | τₒₙ (ms) | τ_{off} (ms) |
|---|---|---|
| 50 | 178 ± 11 | 12 ± 3 |
| 150 | 65 ± 12 | 12 ± 5 |
| 300 | 34 ± 6 | 14 ± 3 |
| 600 | 20 ± 5 | 17 ± 4 |
| 1200 | 10 ± 3 | 13 ± 2 |
| 2400 | 5 ± 1 | 15 ± 1 |

Values are mean±s.d. from n=3 independent experiments. The applied transmembrane potential was +40 mV. The buffer solution contained 300 mM KCl, 10 mM Tris-HCl, 1 mM TCEP, pH 8.

TABLE 7
Mean values of the association (kₒₙ) and dissociation (k_{off})
rate constants of WDR5-Mb4 interactions using an Mb4-tFhuA sensor.

| [WDR5] (nM) | kₒₙ (M⁻¹s⁻¹) × 10⁻⁸ | k_{off} (s⁻¹) |
|---|---|---|
| 50 | 1.1 ± 0.1 | 87 ± 22 |
| 150 | 1.0 ± 0.2 | 88 ± 29 |
| 300 | 1.0 ± 0.2 | 73 ± 18 |
| 600 | 0.9 ± 0.2 | 62 ± 19 |
| 1200 | 0.9 ± 0.3 | 76 ± 8 |
| 2400 | 0.8 ± 0.1 | 67 ± 5 |

Values are mean±s.d. from n=3 independent experiments. The applied transmembrane potential was +40 mV. The buffer solution contained 300 mM KCl, 10 mM Tris-HCl, 1 mM TCEP, pH 8.

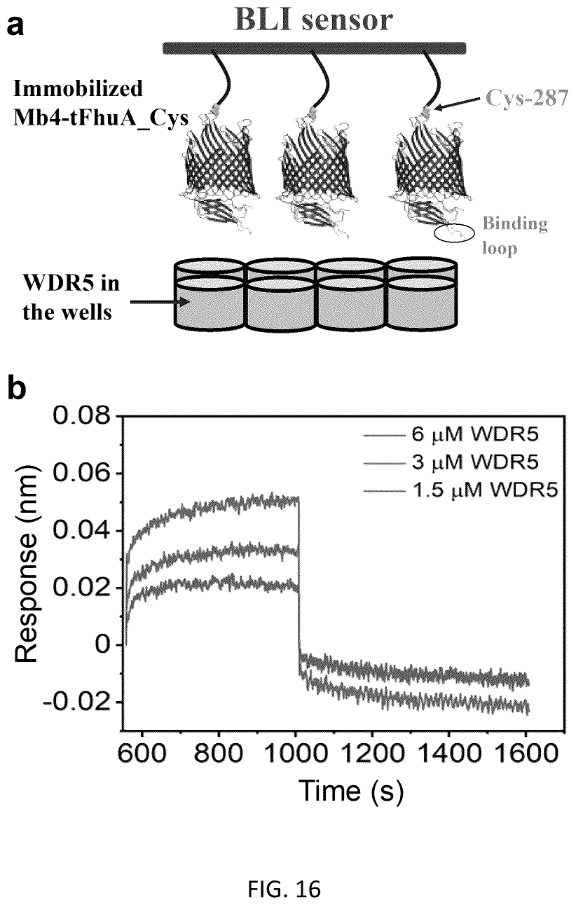

Orthogonal measurements were found to prove the rapid association and dissociation kinetics of WDR5-Mb4 interactions. To validate the fast kinetics recorded with the Mb4-tFhuA sensor, an orthogonal experiment using biolayer interferometry (BLI) was performed. Mb4-tFhuA-containing micelles were immobilized onto the BLI sensor surface using biotin-streptavidin chemistry (Methods; FIG. 16a). Mb4-tFhuA was attached to the sensor surface using a cysteine sulfhydryl engineered on an external loop of tFhuA. Hence, this experimental design mimics in some respect that of a sensing measurement with a Mb4-tFhuA sensor reconstituted into a lipid bilayer. WDR5 was added to different wells at increased concentrations. The association phases were recorded in real time by placing the BLI sensors in WDR-containing wells (FIG. 16b). The dissociation phases were then recorded by placing the same BLI sensors in WDR5-free wells. BLI sensorgrams acquired at various WDR5 concentrations indicate fast association and dissociation phases, confirming the rapid kinetics of WDR5-Mb4 interactions noted with the Mb4-tFhuA sensor (Table 8). Limited time resolution of BLI did not enable us to determine the kinetic parameters of these interactions, highlighting the power of our approach for obtaining quantitative sensitive measurements.

TABLE 8
The association (kₒₙ) and dissociation (k_{off}) rate constants and
equilibrium dissociation constant (K_{D}) of WDR5-Mb4 interactions. kₒₙ
value is the slope of the linear fit in FIG. 3f. k_{off} value is the axis intercept
of the horizontal line fit in FIG. 3g. Here, K_{D} was calculated using the
equation K_{D} + k_{off}/kₒₙ.

| kₒₙ (M⁻¹s⁻¹) × 10⁻⁸ | k_{off} (s⁻¹) | K_{D} (nM) |
|---|---|---|
| 0.83 ± 0.01 | 72.4 ± 3.7 | 872 ± 45 |

Values are mean±s.e.m. The other experimental conditions were the same as those stated in Methods.

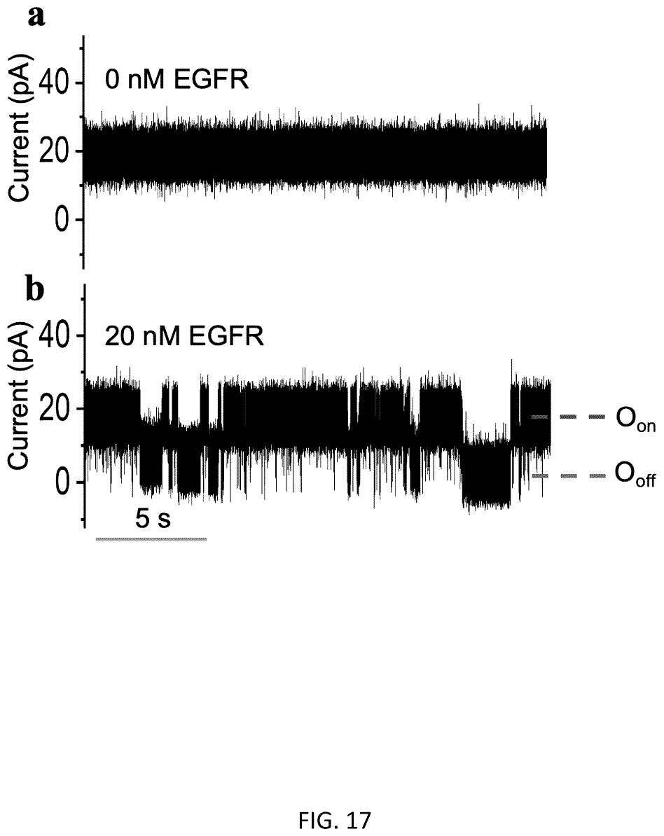

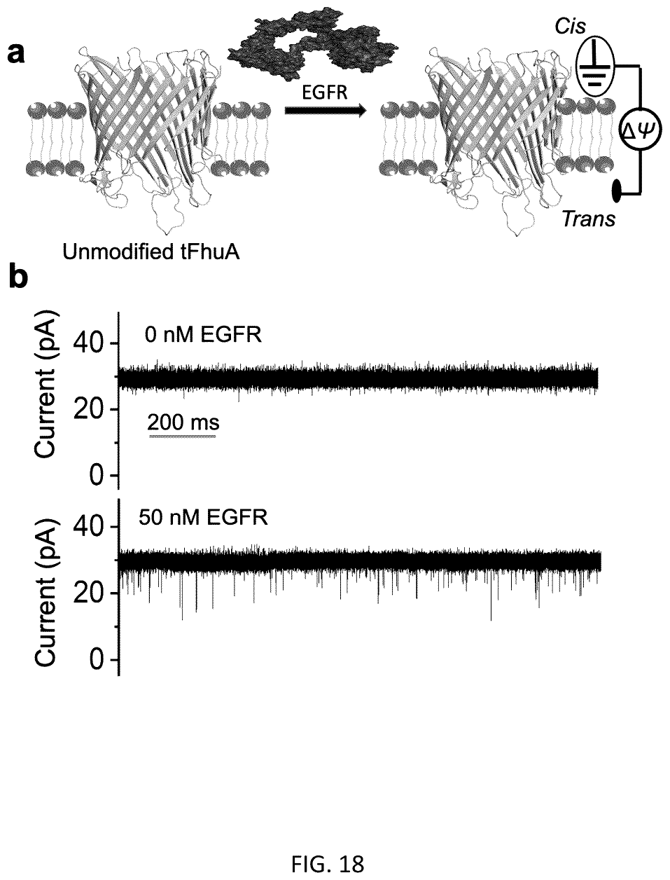

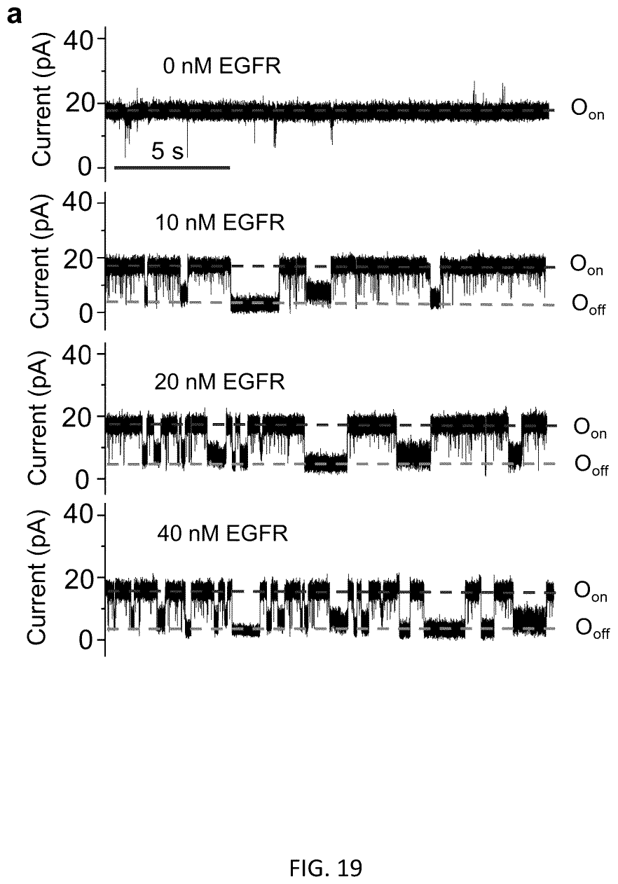

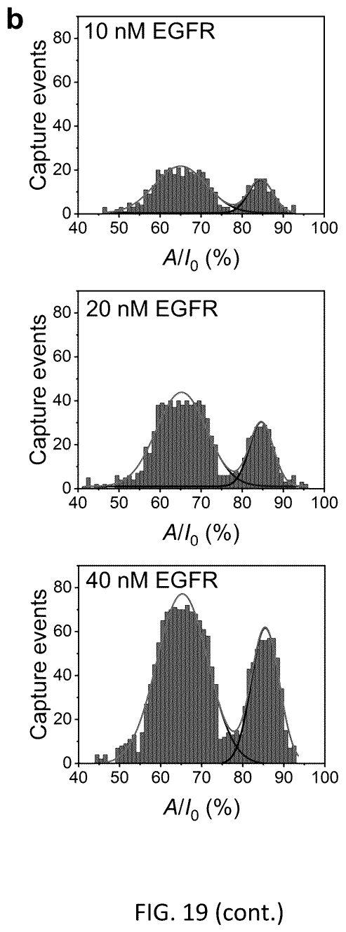

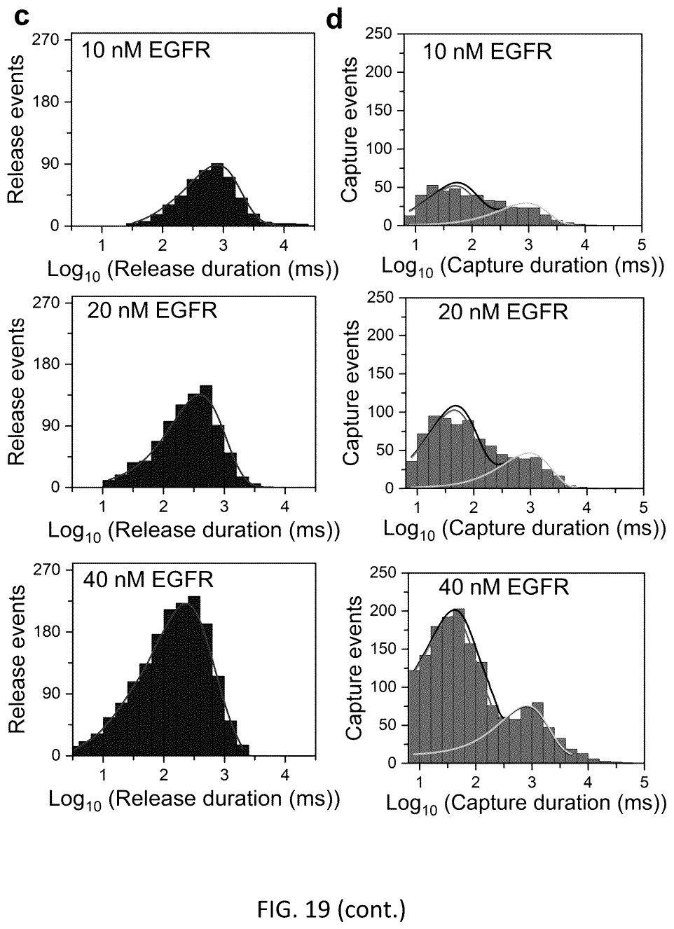

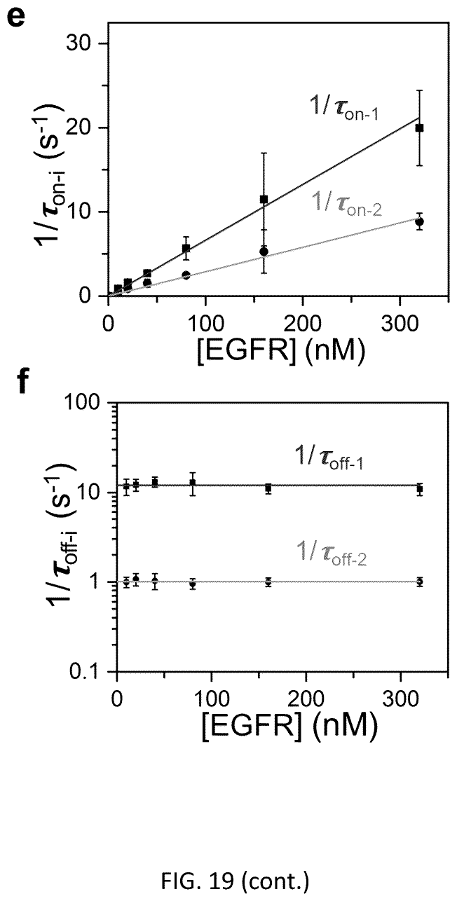

Detection of EGFR biomarker using an Adnectin1-tFhuA. The ectodomain of EGFR is proteolytically released into the bloodstream, allowing this biomarker to be used for screening, diagnosis, and disease progression. Hence, we employed the Adnectin-1 binder against the ectodomain of EGFR. The main benefit of Adnectin-1 is its high affinity with EGFR with a K_{D} of 2 nM. Here, we show real-time detection of EGFR using an Adnectin1-tFhuA sensor. We noted that Adnectin1-tFhuA sensor exhibits some current noise at +40 mV (FIG. 4c), presumably due to the additional flickering of Adnectin1 on top of tFhuA nanopore, which may result from the presence of higher negative charge on the Adnectin1 external surface. However, single-channel electrical traces showed a relatively quiet signature at a lower transmembrane potential of +20 mV (FIG. 19a; FIG. 17). Interestingly, when EGFR was added to the cis compartment, reversible current blockades were observed in a much broader temporal range and with diverse current amplitudes. We interpret that these blockades are produced by specific bindings of EGFR to Adnectin-1. In contrast, we noted only low-amplitude and brief current spikes when EGFR was added to the cis side of a tFhuA sensor-containing bilayer (FIG. 18). When the current amplitudes of individual EGFR-captured events were examined, a two-peak distribution was found (FIG. 19b). For example, at 40 nM EGFR, the normalized current blockades of the two peaks were (65.0±2.1)% and (86.1±1.6)% with the probabilities of 0.72±0.02 and 0.28±0.02, respectively.

Moreover, the relative position and probability of these peaks, for low-amplitude and large-amplitude blockades, were independent of EGFR concentration, [EGFR] (Table 9). EGFR-released (Oₒₙ) and EGFR-captured (O_{off}) durations followed single-peak and double-peak event distributions (FIG. 19cd), respectively, as judged by the maximum likelihood method and logarithm likelihood ratio (LLR) tests. Hence, our statistical analyses revealed two subpopulations of binding events (FIG. 19d), the short-lived and long-lived events, whose EGFR capture durations were ˜80 ms and ˜1 s, respectively (Tables 10-12). Interestingly, the probabilities of short-lived durations (τ_{off-1}), P₁, are close to those of low-amplitude current blockades (Tables 9-10). This outcome suggests two distinct mechanisms of binding of Adnectin1 to EGFR, which are correlated with both the extent of the current amplitude (A/I₀) of EGFR-captured events and their duration (τ_{off}).

TABLE 9
Values of the mean normalized amplitude of EGFR-produced current
blockades, A/I₀, for two binding events at various [EGFR] values.
Here, I₀ and A denote the single-channel current of the EGFR-released
substate and the current amplitude of EGFR-produced current blockade,
respectively. A/I₀ was then converted into percentage. P₁ and P₂
represent the probabilities of two distinct current blockades. A
representative event histogram of normalized current amplitudes
is shown in FIG. 4b.

| EGFR [nM] | P₁ | A₁/I₀ (%) | P₂ | A₂/I₀ (%) |
|---|---|---|---|---|
| 10 | 0.72 ± 0.01 | 63.8 ± 1.5 | 0.27 ± 0.01 | 85.1 ± 2.3 |
| 20 | 0.73 ± 0.02 | 64.9 ± 1.1 | 0.27 ± 0.02 | 84.4 ± 3.1 |
| 40 | 0.72 ± 0.02 | 65.0 ± 2.1 | 0.28 ± 0.02 | 86.1 ± 1.6 |
| 80 | 0.75 ± 0.03 | 67.3 ± 6.9 | 0.25 ± 0.03 | 85.2 ± 1.5 |
| 160 | 0.74 ± 0.04 | 66.6 ± 5.2 | 0.26 ± 0.04 | 86.8 ± 4.0 |
| 320 | 0.76 ± 0.04 | 66.0 ± 3.6 | 0.24 ± 0.04 | 87.3 ± 5.8 |

Values of the normalized current blockades are mean±s.d. from n=3 independent experiments.

TABLE 10
The probability distribution of the two binding
events of Adnectin1-EGFR interactions.

| EGFR [nM] | P₁ | P₂ |
|---|---|---|
| 10 | 0.65 ± 0.05 | 0.34 ± 0.05 |
| 20 | 0.65 ± 0.02 | 0.34 ± 0.02 |
| 40 | 0.64 ± 0.05 | 0.35 ± 0.05 |
| 80 | 0.69 ± 0.03 | 0.30 ± 0.03 |
| 160 | 0.68 ± 0.02 | 0.31 ± 0.02 |
| 320 | 0.69 ± 0.05 | 0.31 ± 0.05 |

Values are mean±s.d. from n=3 independent experiments. P₁, and P₂ are the probabilities of the short- and long-lived EGFR-captured events (Table 9), respectively. The applied transmembrane potential was +20 mV. The buffer solution contained 300 mM KCl, 10 mM Tris-HCl, pH 8.0. These events were differentiated by the EGFR-captured duration. Individual experimental values were derived using event histograms in ClampFit (Axon) and fits in a semilogarithmic representation. The maximum likelihood method and logarithm likelihood ratio (LLR) tests were used for all fits to determine the best multi-exponential distribution model (Methods).

TABLE 11
Mean values of durations of the short-lived (τ_{off-1})
and long-lived (τ_{off-2}) EGFR-captured events.

| EGFR [nM] | τoff−1 (ms) | τoff−2 (ms) |
|---|---|---|
| 10 | 88 ± 19 | 1016 ± 135 |
| 20 | 83 ± 26 | 950 ± 150 |
| 40 | 76 ± 10 | 1000 ± 199 |
| 80 | 81 ± 23 | 1060 ± 140 |
| 160 | 91 ± 11 | 1013 ± 111 |
| 320 | 92 ± 13 | 1005 ± 109 |

Values are mean±s.d. from n=3 independent experiments. The applied transmembrane potential was +20 mV. The buffer solution contained 300 mM KCl, 10 mM Tris-HCl, pH 8.0. All histogram fittings were conducted using a semilogarithmic representation. The maximum likelihood method^{4, 5} and logarithm likelihood ratio (LLR) tests⁶⁻⁸ were used for all fits to determine the best multi-exponential distribution model (Methods).

TABLE 12
Mean values of the cumulative τₒₙ, τₒₙ₋₁ and τₒₙ₋₂
EGFR-released events of EGFR-Adnectin1 interactions.

| EGFR [nM] | τon (S) | τon − 1 (S) | τon − 2 (S) |
|---|---|---|---|
| 10 | 0.79 ± 0.07 | 1.2 ± 0.1 | 2.5 ± 0.2 |
| 20 | 0.41 ± 0.06 | 0.62 ± 0.07 | 1.2 ± 0.2 |
| 40 | 0.24 ± 0.04 | 0.37 ± 0.05 | 0.70 ± 0.18 |
| 80 | 0.12 ± 0.02 | 0.18 ± 0.04 | 0.41 ± 0.05 |
| 160 | 0.07 ± 0.03 | 0.10 ± 0.04 | 0.21 ± 0.08 |
| 320 | 0.035 ± 0.005 | 0.05 ± 0.01 | 0.11 ± 0.01 |

Values are mean±s.d. from n=3 independent experiments. The applied transmembrane potential was +20 mV. The buffer solution contained 300 mM KCl, 10 mM Tris-HCl, pH 8.0. Subscripts “i” 1 and 2 stand for short- and long-lived EGFR capture events, respectively. τₒₙ are mean values of the single-exponential distributions of EGFR-released duration histograms. τₒₙ₋₁=τₒₙ/P₁, where P₁ is the event probability of the short-lived events for each experiment. τₒₙ₋₂=τₒₙ/P₂, where P₂ is the event probability of the long-lived events for each experiment. The mean values of those probabilities are listed in Table 8. All histogram fits were conducted using a semilogarithmic representation. The maximum likelihood method^{4, 5} and logarithm likelihood ratio (LLR)^{6, 7, 9} tests were used for all fits to determine the best multi-exponential distribution model (Methods).

Furthermore, the frequency of EGFR-captured was amplified by increasing the EGFR concentration, [EGFR] (FIG. 19a). The event frequencies of short-lived and long-lived EGFR-captured events, in the form of 1/τₒₙ₋₁ and 1/τₒₙ₋₂, respectively, were linearly dependent on [EGFR] (FIG. 19e; Table 13). Here, τₒₙ₋₁ and τₒₙ₋₂ are the release (e.g., interevent) durations of the short-lived and long-lived EGFR-captured events, respectively (Table 12). Again, the dissociation constants of the short-lived (k_{off-1}) and long-lived (k_{off-1}) current blockades were independent of [EGFR] (FIG. 19f; Table 14). Therefore, the single-molecule sensing approach provides both the identity and quantity of each substate of Adnectin1-EGFR interactions. The association rate constants, kₒₙ₋₁ and kₒₙ₋₂ (mean±s.e.m), were (6.62±0.21)×10⁷ M⁻¹s⁻¹ and (2.89±0.10)×10⁷ M⁻¹s⁻¹, respectively. The dissociation rate constants, k_{off-1} and k_{off-1} (mean±s.e.m), were 12.0±0.4 s⁻¹ and 1.01±0.01 s⁻¹, respectively (Table 15). These values yield the equilibrium dissociation constants of the short-lived and long-lived current blockades, K_{D-1} and K_{D-2} (mean±s.e.m), respectively, of 181±8 nM and 34±2 nM, respectively.

TABLE 13
Mean values of the association rate constants corresponding
to the short-lived (kₒₙ₋₁) and long-lived (kₒₙ₋₂)
current blockades produced by EGFR-Adnectin1 interactions.

| EGFR [nM] | kₒₙ₋₁ (M⁻¹s⁻¹) × 10⁻⁷ | kₒₙ₋₂ (M⁻¹s⁻¹) × 10⁻⁷ |
|---|---|---|
| 10 | 8.8 ± 1.0 | 4.1 ± 0.3 |
| 20 | 8.1 ± 1.1 | 4.4 ± 0.7 |
| 40 | 6.8 ± 0.9 | 3.8 ± 1.2 |
| 80 | 7.1 ± 1.7 | 3.1 ± 0.4 |
| 160 | 7.2 ± 3.5 | 3.3 ± 1.6 |
| 320 | 6.2 ± 1.4 | 2.8 ± 0.3 |

Values are mean±s.d. from n=3 independent experiments. The applied transmembrane potential was +20 mV. The buffer solution contained 300 mM KCl, 10 mM Tris-HCl, pH 8.0.

TABLE 14
Mean values of the dissociation rate constants corresponding
to the short-lived (k_{off-1}) and long-lived (k_{off-2})
EGFR-captured events of EGFR-Adnectin1 interactions.

| EGFR [nM] | k_{off-1} (s⁻¹) | k_{off-2} (s⁻¹) |
|---|---|---|
| 10 | 12 ± 2 | 1.0 ± 0.1 |
| 20 | 12 ± 2 | 1.1 ± 0.2 |
| 40 | 13 ± 2 | 1.0 ± 0.2 |
| 80 | 13 ± 4 | 1.0 ± 0.1 |
| 160 | 11 ± 1 | 1.0 ± 0.1 |
| 320 | 11 ± 2 | 1.0 ± 0.1 |

Values are mean±s.d. from n=3 independent experiments. The applied transmembrane potential was +20 mV. The buffer solution contained 300 mM KCl, 10 mM Tris-HCl, pH 8.0.

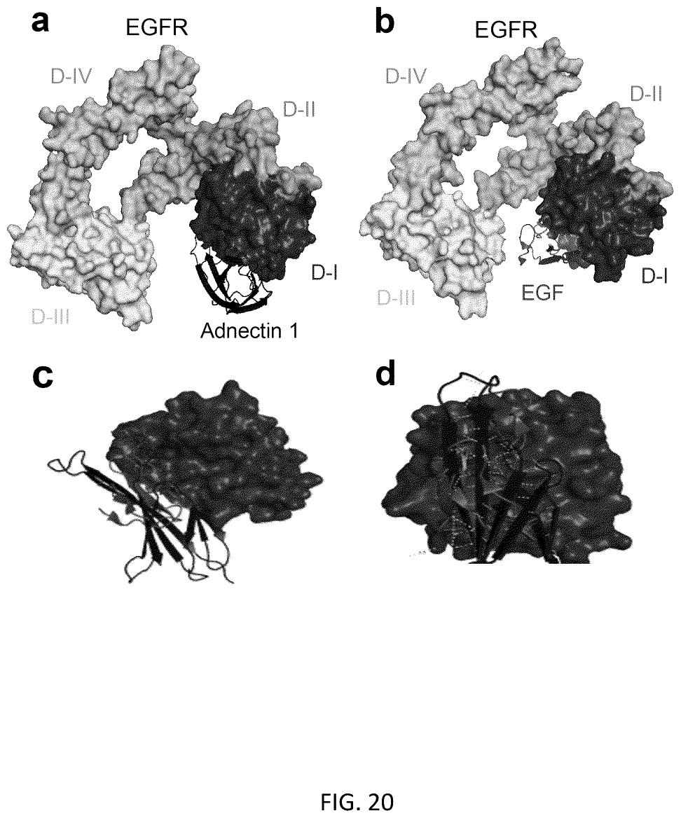

The EGFR structure in the EGFR/EGF complex (1NQL.pdb) is mostly similar that of EGFR in the EGFR-Adnectin1 complex (3QWQ.pdb) and is believed to be an inactive form of the receptor (FIG. 20ab). Adnectin1 and EGF bind to the EGFR domain D-I with a highly overlapping binding surface (FIG. 20cd). It is well established that EGFR is a remarkably adaptable molecule where the domains D-I and D-III are relatively rigid, whereas the domains D-II and D-IV can adopt multiple conformations that place domain D-III differently in relation to domain D-I. We speculate that such distinct conformers of a flexible EGFR may likely be responsible for the multimodal protein recognition of EGFR by Adnectin1. The extended time bandwidth of our measurements facilitated the detection and quantification of conformational binding substates of the EGFR-Adnectin1 complex that are actually hidden in ensemble measurements or in low-resolution single-molecule measurements. Earlier studies using the resistive pulse-technique have reported this kind of bimodal conformational transitions in the case of dihydrofolate reductase (DHFR) enzyme.

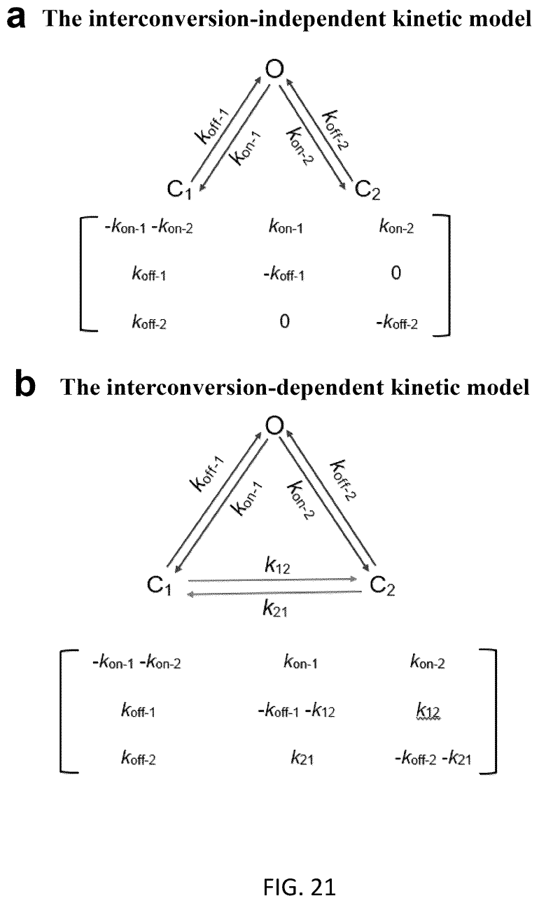

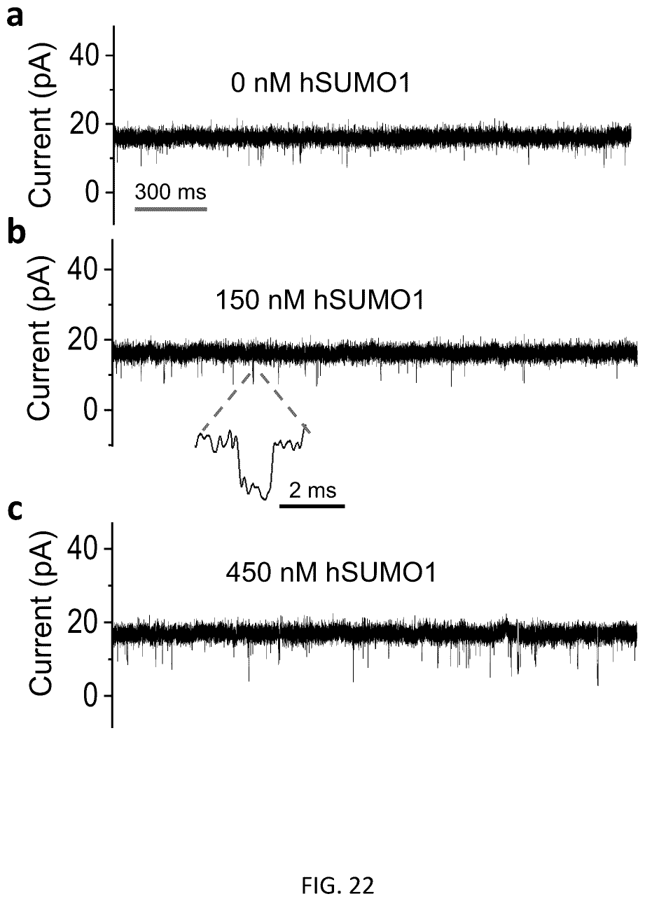

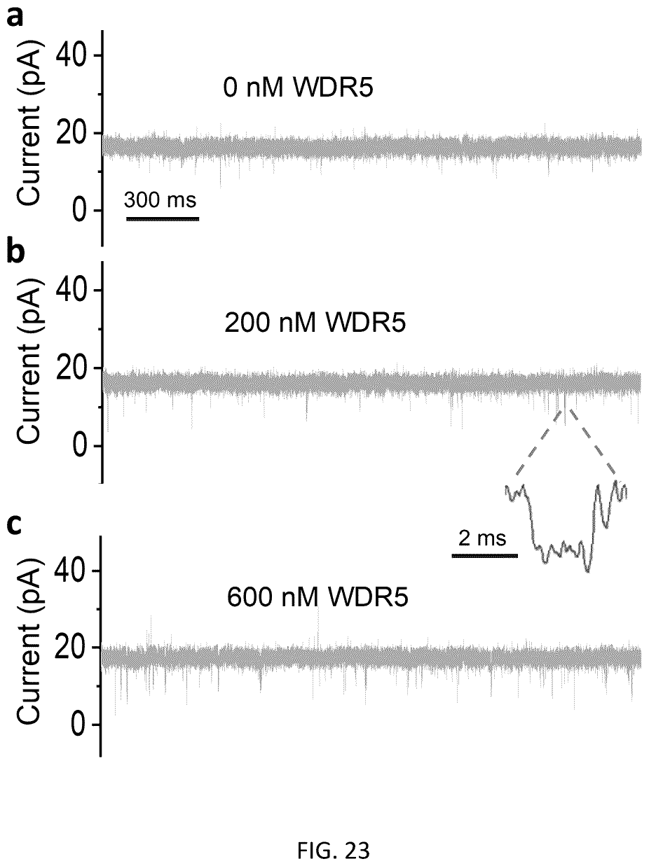

Next, an investigation was performed to determine whether these reversible current transitions may also involve transitions taking place between the two EGFR-captured substates, “1” and “2”. Hence, a related question is whether a kinetic model including interconversion transitions between these capture substates would more accurately reflect experimentally determined rate constants. An interconversion-dependent kinetic model was then developed, encompassing two supplementary rate constants between EGFR-captured substates, k₁₂ and k₂₁ (Table 16, FIG. 21). At a confidence level C >0.95, the interconversion-dependent kinetic model was not statistically superior over those corresponding to an interconversion-independent kinetic model, as indicated by the LLR test. Finally, to test the reactivity crosscheck of our sensors, electrical traces of Adnectin1-tFhuA were recorded in the presence of either hSUMO1 (FIG. 22) or WDR5 (FIG. 23). In both cases, very short-lived and low-amplitude current blockades were noted. These blockades rather resemble those typically found in the case of nonspecific interactions of folded proteins with the cis opening of tFhuA (FIGS. 10, 9, and 18). This finding proves that our Adnectin1-tFhuA sensor is highly specific to EGFR.

TABLE 15

| kₒₙ₋₁ | kₒₙ₋₂ | | | | |
|---|---|---|---|---|---|
| (M⁻¹s⁻¹) × | (M⁻¹s⁻¹) × | k_{off-1} | k_{off-2} | K_{D-1} | K_{D-2} |
| 10⁻⁷ | 10⁻⁷ | (s⁻¹) | (s⁻¹) | (nM) | (nM) |
| 6.62 ± 0.21 | 2.89 ± 0.10 | 12.0 ± 0.4 | 1.01 ± 0.01 | 181 ± 8 | 34 ± 2 |

Values are mean±s.e.m. The other experimental conditions were the same as those stated in Methods. The association and dissociation rate constants and the equilibrium dissociation constant, K_{D}. kₒₙ₋₁ and kₒₙ₋₂ values are the slopes of the linear fits in FIG. 4e. k_{off-1} and k_{off-2} values are the axis intercepts of the horizontal line fits in FIG. 4f K_{D-1} and K_{D-2} are the equilibrium dissociation constants of the short-lived and long-lived current blockades. Here, K_{D} was calculated using the equation K_{D}=k_{off}/kₒₙ.

TABLE 16
Transition rate constants obtained for the
interconversion-dependent kinetic model.

| [EGFR] (nM) | k_{off-1} (s⁻¹) | k₁₂ (s⁻¹) | k₂₁ (s⁻¹) | k_{off-2} (s⁻¹) |
|---|---|---|---|---|
| 20 | 15.6 ± 2.8 | 1.08 ± 0.20 | 0.46 ± 0.03 | 0.39 ± 0.09 |

Raw single-channel traces were analyzed event using MATLAB (MathWorks, Natick, MA). The EGFR concentration was 20 nM. Values were provided as mean±s.d. (n=3), where n is the number of independently reconstituted nanopores. The association rate constants kₒₙ₋₁ and kₒₙ₋₂ obtained by this model were the same as those determined by the interconversion-independent kinetic model. The interconversion-independent and interconversion-dependent kinetic models are illustrated in FIG. 21.

Single-molecule detection of a protein biomarker in a biofluid.

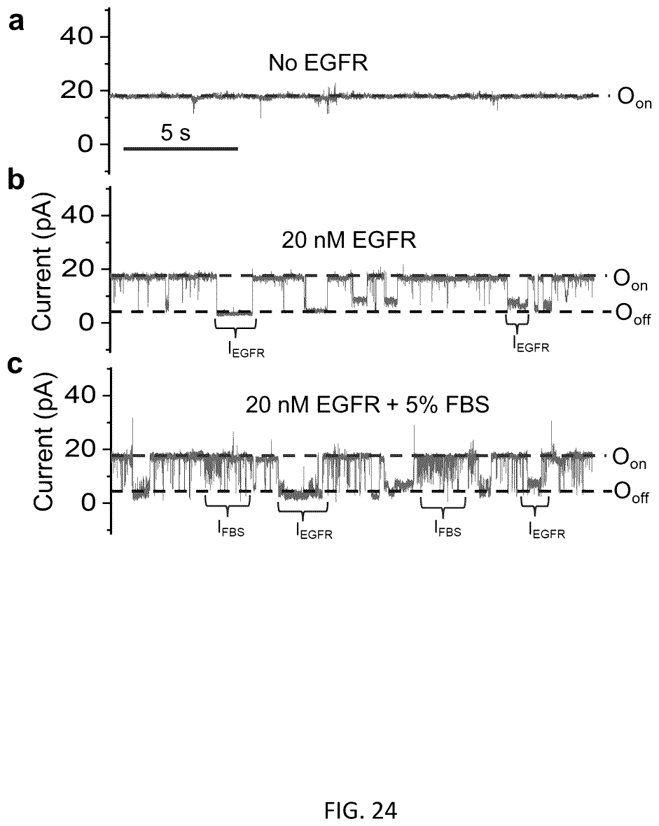

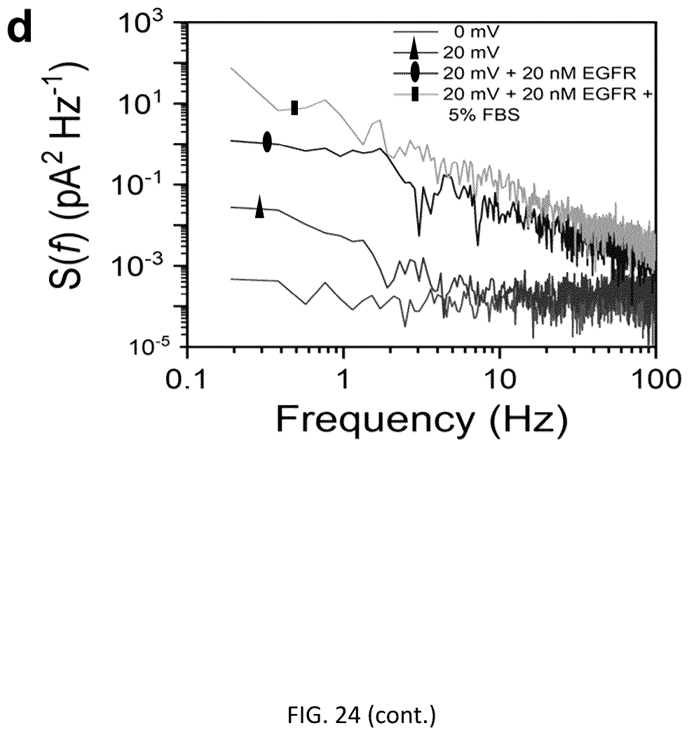

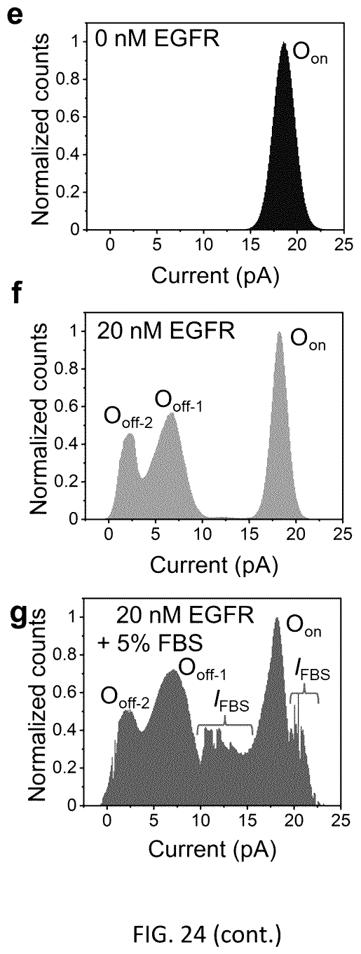

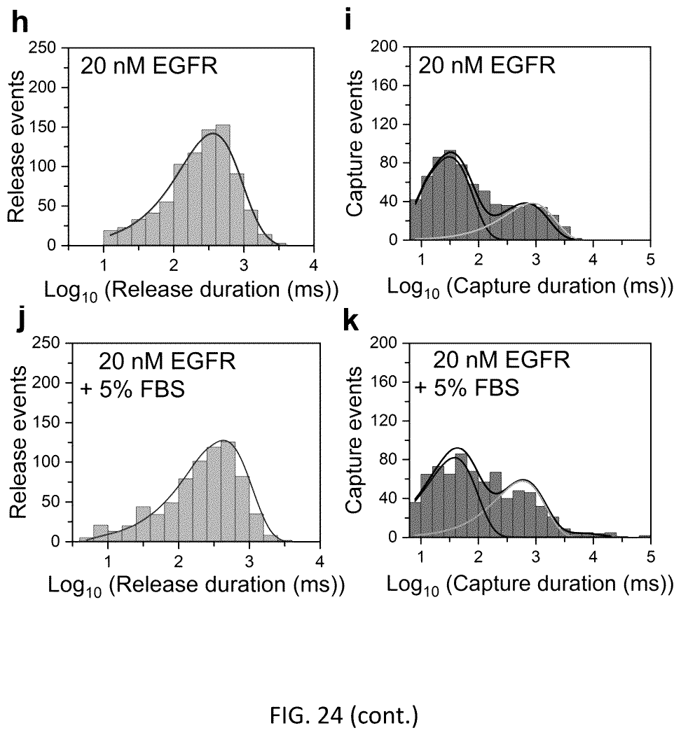

These sensors were challenged in the presence of 5% fetal bovine serum (FBS) to examine the stability of this system in a challenging environment and the ability to distinguish analyte-capture events from other nonspecific transitions of the serum constituents. The serum threshold for the soluble EGFR ectodomain level is 45 ng/ml (˜112 nM). The tumor state can be evaluated at EGFR levels significantly exceeding this threshold. FIG. 24a and FIG. 24b show a representative signature of Adnectin1-tFhuA in the absence and presence of 20 nM EGFR, respectively. However, the addition of 5% FBS decorated the standard signature of EGFR-captured events with brief current spikes in the low-millisecond range (FIG. 24c). An analysis of the power spectral density (PSD) of current fluctuations revealed transition from white noise in the absence of EGFR to 1/f flicker noise in the presence of EGFR (FIG. 24d). In addition, the noise amplitude is slightly amplified when FBS was present in the chamber. This outcome suggests low-frequency equilibrium fluctuations in the local mobility and density of charges at the tip of the sensor. The brief FBS-induced current fluctuations had a lower-amplitude around the open Oₒₙ substate (FIG. 24e-g), indicating that these may result from trafficking moieties of serum constituents at the cis opening of the Adnectin1-tFhuA sensor (FIG. 1). An extensive statistical analysis of the current blockades corresponding to the levels O_{off-1} and O_{off-2} confirmed the presence of two EGFR-captured event types in the presence of FBS (FIG. 24h-k; Tables 17-18). No statistically significant impact of FBS was noted on the k_{off-1} and k_{off-2}, but small changes, within the same order of magnitude, on the kₒₙ₋₁ and kₒₙ₋₂. (Table 19). These changes are perhaps a result of the interference of serum constituents with the binding interfaces of EGFR and Adnectin1. The mean duration of long-lived EGFR-induced current blockades is τ_{off-2}=0.95±0.08 s, much longer than the brief millisecond-timescale FBS-induced closures. Under these conditions, we determined a corresponding τₒₙ₋₂ of 1.65±0.50 s. Using a kₒₙ₋₂ of (2.89±0.17)×10⁷ M⁻¹s⁻¹ in the absence of FBS, we can evaluate the EGFR concentration in the serum sample, [EGFR], using the equation [EGFR]=1/(τₒₙ₋₂/kₒₙ₋₂). Employing these values, we determined an [EGFR] of 22.2±5.9 nM in the serum sample, near the actual concentration of 20 nM. We judge that the distinction between the measured and actual values was brought about by the interference of serum constituents with the sensor.

TABLE 17
The probability distribution of the two binding events
noted with EGFR-Adnectin1 interactions in the absence
and presence of fetal bovine serum (FBS) (5%).

| 5% FBS | P1 | P2 |
|---|---|---|
| − | 0.65 ± 0.04 | 0.35 ± 0.04 |
| + | 0.62 ± 0.06 | 0.38 ± 0.06 |

Values are mean±s.d. from n=3 independent experiments. P₁ and P₂ are the probabilities of the short- and long-lived EGFR-captured events, respectively. The other experimental conditions were the same as those stated in Methods. These events were differentiated by the EGFR-captured duration. Individual experimental values were derived using event-list histograms in ClampFit (Axon) and their fits in a semilogarithmic representation. The maximum likelihood method and logarithm likelihood ratio (LLR) tests were used for all fits to determine the best multi-exponential distribution model (Methods).

TABLE 18
Mean values of the τₒₙ and τ_{off} constants of EGFR-Adnectin1
interactions in the absence and presence of FBS.

| 5% FBS | τₒₙ (s) | τₒₙ₋₁ (s) | τₒₙ₋₂ (s) | τ_{off-1} (s) | τ_{off-2} (s) |
|---|---|---|---|---|---|
| − | 0.40 ± | 0.61 ± | 1.2 ± | 0.074 ± | 0.97 ± |
| | 0.05 | 0.043 | 0.1 | 0.027 | 0.09 |
| + | 0.61 ± | 0.98 ± | 1.7 ± | 0.062 ± | 0.93 ± |
| | 0.09 | 0.05 | 0.5 | 0.023 | 0.14 |

Values in are mean±s.d. (− is the absence of FBS from n=3 independent experiments and + is the presence of FBS from n=3 independent experiments). The applied transmembrane potential was +20 mV. The buffer solution contained 20 nM EGFR, 300 mM KCl, 10 mM Tris-HCl, pH 8.0.

TABLE 19
Mean values of kₒₙ and k_{off} kinetic rate constants of EGFR-
Adnectin1 interactions in the absence and presence of FBS.

| | kₒₙ₋₁ | kₒₙ₋₂ | k_{off-1} | k_{off-2} |
|---|---|---|---|---|
| FBS | (M⁻¹s⁻¹) × 10⁻⁷ | (M⁻¹s⁻¹) × 10⁻⁷ | (s⁻¹) | (s⁻¹) |
| − | 8.84 ± 0.58 | 4.41 ± 0.72 | 15.12 ± 6.65 | 1.04 ± 0.09 |
| + | 5.13 ± 0.25 | 3.22 ± 0.91 | 18.04 ± 8.44 | 1.08 ± 0.15 |

Values are mean±s.d. (− is the absence of FBS from n=3 independent experiments and + is the presence of FBS from n=3 independent experiments). The applied transmembrane potential was +20 mV. The buffer solution contained 20 nM EGFR, 300 mM KCl, 10 mM Tris-HCl, pH 8.0.

Distinctive Outcomes with Monobody-Based Sensors

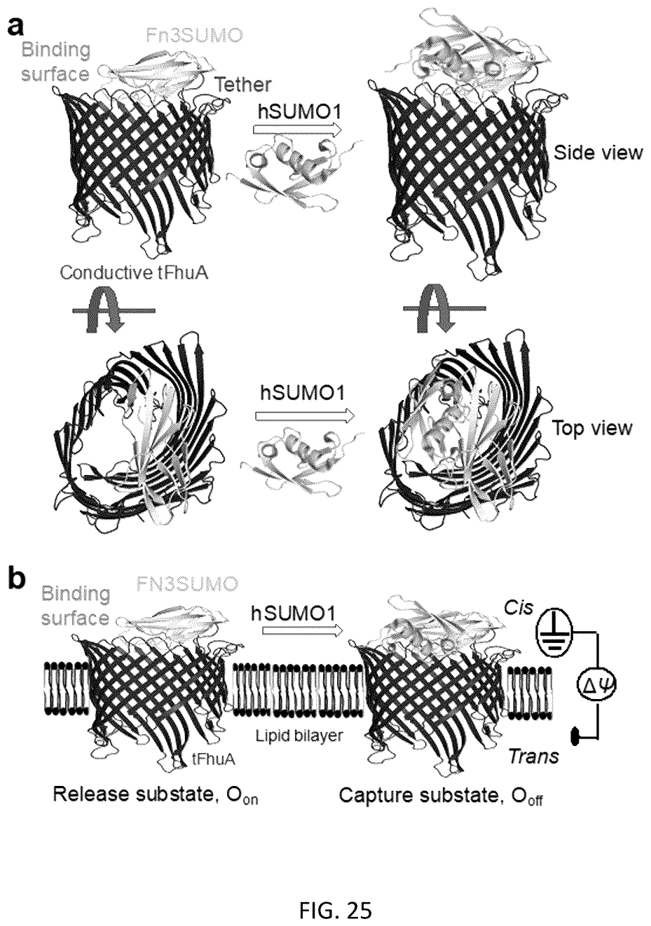

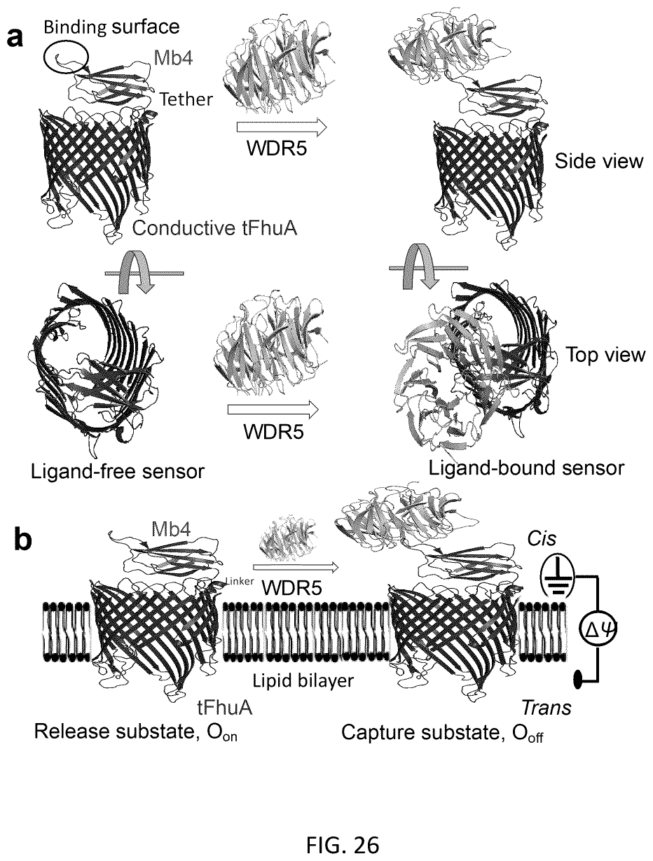

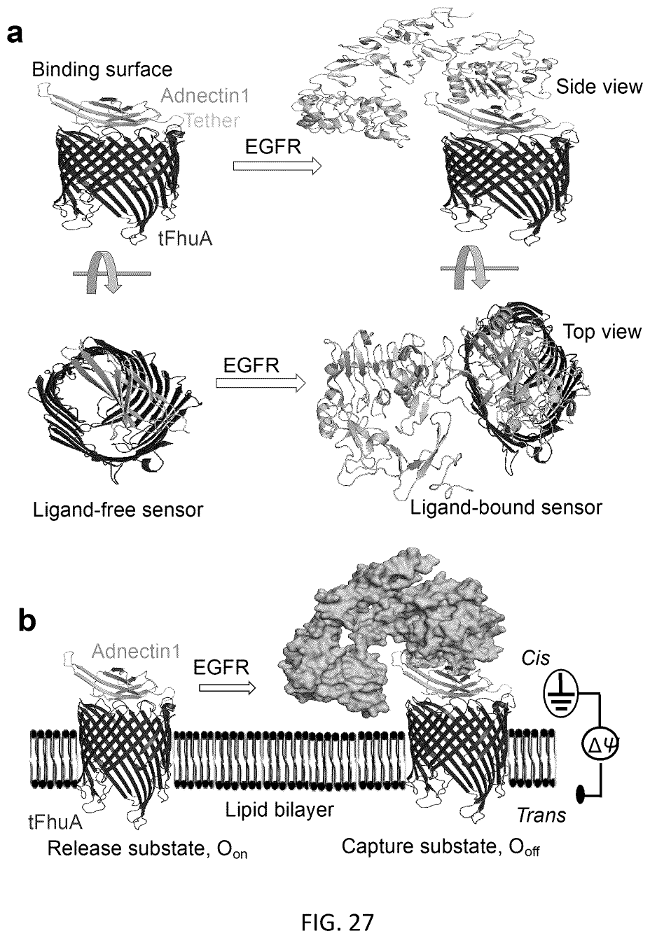

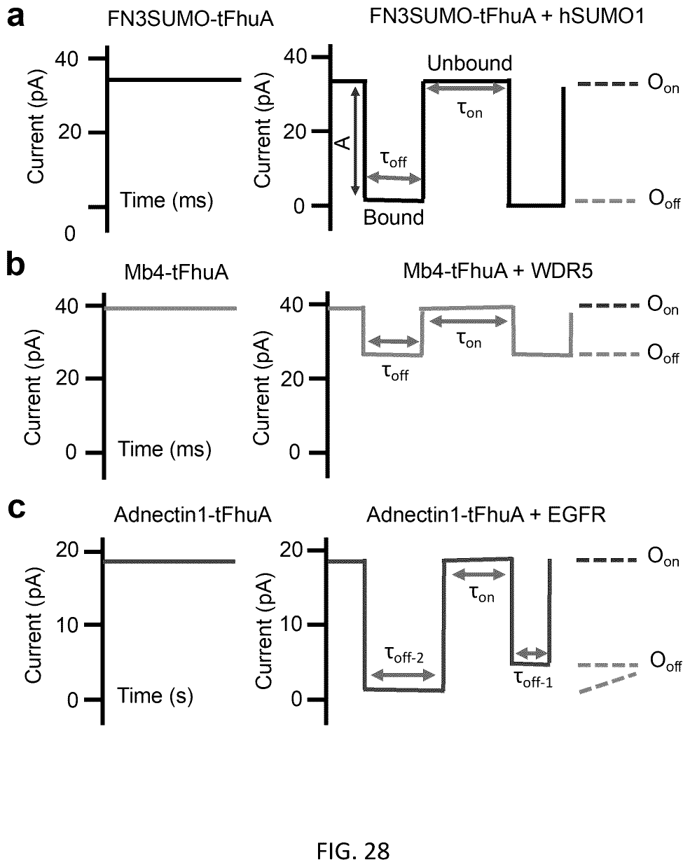

The present invention provides a detailed signature analysis of single-molecule protein detection of three analytes using three nanopore sensors that share a common modular architecture, but they differ by their binding surface (FIGS. 25-27). Furthermore, the Alphafold2 procedure predicted closely similar orientations of monobodies with respect to the central axis of tFhuA. Because the protein analytes and their complexes with the specific monobodies drastically vary in size, charge, and complexity, distinct current blockades are noted in each case (FIG. 28). This experimental observation is consistent of the modeling structures of monobody-analyte complexes at the tip of nanopore sensors. For example, WDR5 interacts with a distal loop of Mb4 and away from the tFhuA pore opening (FIG. 26), suggesting only a modest current blockade upon the WDR5-Mb4 complex formation. In accord with this expectation, it is noted that low-amplitude current blockades made by WDR5-captured events (Table 20). In contrast, the conformation complexity and structural properties of the hSUMO1-FN3SUMO and EGFR-Adnectin1 dimers upon their formation at the tip of the sensor indicated a potentially large ionic flow block. This outcome is in accordance with the recorded signatures. In addition, distinct single-molecule kinetic signatures were probed of each analyte in the absence of the steric restrictions, otherwise imposed by the confinement of detection within the nanopore lumen (FIG. 28, Table 20). Similar capture durations were noted for hSUMO1 and WDR5, yet these protein analytes exhibited distinguishable current-amplitude signatures, showing the strength of the present sensor design. These unique characteristics of protein detections using externally engineered complex binding interfaces culminated with the discovery of a bimodal protein recognition of EGFR.

TABLE 20
Comparison of capture durations and current
blockades of the protein analytes.

| | Capture | Normalized current |
|---|---|---|
| Protein | duration | blockades |
| analytes | (ms) | A/I₀ (%) |
| hSUMO1 | 13 ± 1 | 92 ± 1 |
| WDR5 | 14 ± 1 | 14 ± 1 |
| EGFR | 1010 ± 14 | 65 ± 2 |
| | 85 ± 3 | 86 ± 1 |

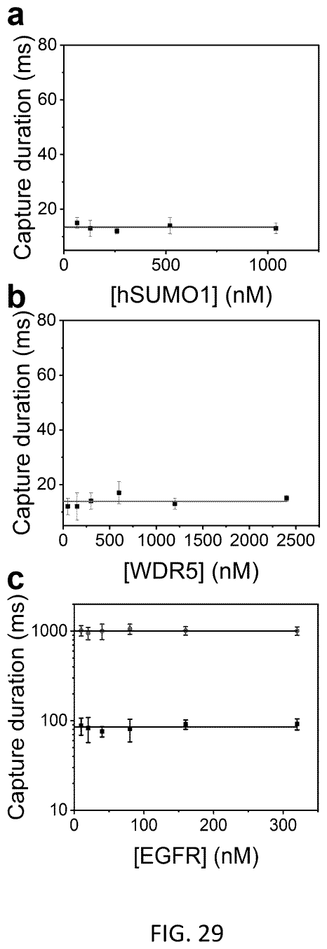

Capture durations represent mean±s.e.m using the fits in FIG. 29. Values of normalized current blockades are mean±s.d. from n=3 independent experiments. These values are determined at concentrations of 65 nM hSUMO1, 50 nM WDR5, and 40 nM EGFR.

Validation of the Monobody-Based Sensors

To validate these monobody-based sensors, we examined the binding affinity of detergent-refolded sensors with their cognate protein analytes using steady-state FP anisotropy. If the labeled protein analyte interacts with the corresponding monobody-containing nanopore sensor, then its tumbling rate (e.g., the coefficient of rotational diffusion) would decrease, increasing the FP anisotropy. In accord with our expectation, the FP anisotropy substantially increased at elevated sensor concentrations (FIG. 29). On the contrary, the presence of tFhuA did not produce any change in the baseline value of the FP anisotropy in all cases, confirming no interaction between labelled proteins and sensor-containing detergent micelles. The calculated K D values of hSUMO1 and WDR5 with their respective nanopore sensors were 186±16 and 455±59 nM, respectively, which agrees with the outcome of single-channel electrical recordings of these sensors (Tables 5, 8). EGFR is not suitable for this assay, because of its large molecular weight, so its tumbling rate is longer than the fluorescence lifetime of most fluorophores. However, the K_{D} determined for the long-lived EGFR-captured events using Adnectin1-tFhuA sensor is in accord with the result of a previously reported study (Table 15). It should be mentioned that the restrain of one binding partner to a surface can decrease the binding affinity up to one order of magnitude. Hence, this explains a slightly weaker binding interaction with the immobilized nanopore sensor protein onto a lipid bilayer than that value measured in solution by steady-state FP spectroscopy.

Advantages of this Class of Protein Sensors and its Implications in Nanobiotechnology

The new class of nanopore sensors made of a single-polypeptide chain protein according to the present invention circumvents the necessity of tedious purification steps, otherwise needed for multimeric nanopore sensors. The overall architecture of the sensors can be maintained while changing the binding interface of the antibody-mimetic binder. This way, such an approach substantially expands the utility of these sensing elements for numerous protein biomarkers while preserving their high specificity and sensitivity. This critical benefit is facilitated by the genetically encoded nature of these sensors so that they can create combinatorial libraries of tethered binders. In addition, there is no fundamental limitation or technical challenge in replacing the monobody with another small protein scaffold binder, such as an affibody or an anticalin. Furthermore, the main advantages of using antibody-mimetic proteins include strong binding affinities with different epitopes, straightforward expression and purification procedures, lack of disulfide bonds, and high thermodynamic stability. Fortuitously, in the current sensor configuration, monobodies orient about 90° with respect to the central axis of tFhuA, allowing direct electrical detection of analyte bindings without needing an adapter. Because most antibody-mimetic scaffolds are developed against the native binding sites of proteins, our sensors can be potentially utilized for the screening of specific libraries of small-molecule inhibitors. These sensors can operate in challenging biofluids at clinically relevant concentration ranges and with an extended time bandwidth. In this process, the biomarker-induced events are unambiguously distinguished from other nonspecific bindings of biofluid constituents. Therefore, with further development these synthetic nanopore sensors can be integrated into nanofluidic devices and coupled with high-throughput technologies for biomarker profiling in biomedical diagnostics.

Methods

Synthetic Gene Construction.

Three derivatives of wild-type fibronectin type-III (10FN3) were used for the development of these sensors. The cDNA sequences of these 10fn3 genes, namely fn3sumo, mb4, and adnectin 1, were fused to the 5′ end of the tfhua gene via a (GGS)₂-encoding linker by a restriction-free cloning method. The cDNA sequences of Mb4 and Adnectin1 were synthesized by Eurofins Genomics (Louisville, KY) and Integrated DNA Technologies (IDT, Coralville, Iowa), respectively. The construction of the fn3sumo gene was made based on ySMB9. The cDNA sequence of all three fibronectin derivatives was first amplified using Q5 high-fidelity DNA polymerase (New England BioLabs, Ipswich, MA) from their respective template DNA. PCR products were separated on 1% agarose gel and purified using a Gel extraction kit (Promega, CA). Sequences of forward and reverse primers are listed in Table 21. Amplified products of fn3sumo and mb4 genes were then fused to the 5′ end of tfhua cloned in pPR-IBA1 plasmid (IBA, Goettingen, Germany). The adnectin1 gene was joined at the 5′ end of the tfhua gene in pET28a (EMD Millipore, Burlington, MA). The pET28-tFhuA plasmid was constructed by inserting the gene between BamHI and XhoI restriction sites after amplification with forward and reverse primers of tFhuA (Table 21). All the gene sequences were verified by sequencing (MCLab, San Francisco, CA). The pET11a-hSUMO1 was kindly provided by Fauke Mechior (Addgene plasmid #53138).

TABLE 21
List of primers used in this study.

| Primer name | Sequences (5′-3′) | ID |
|---|---|---|
| FN3SUMO_ | CTTTAAGAAGGAGAT | SEQ ID NO: 2 |
| for | ATACAAATGGGTAGC | |
| | CCGAGCGTTCCGGGC | |
| FN3SUMO_ | CTGAACTTCTTTCAG | SEQ ID NO: 3 |
| rev | GCTGCCGCCGCTGCC | |
| | GCCGGTGGTAACGCT | |
| | AACGCTGC | |
| Mb4_for | CTTTAAGAAGGAGAT | SEQ ID NO: 4 |
| | ATACAAATGGGATCT | |
| | TCTGTTCCGACC | |
| Mb4_rev | CTGAACTTCTTTCAG | SEQ ID NO: 5 |
| | GCTGCCGCCGCTGCC | |
| | GCCGGTACGGTAGTT | |
| | AATCGAG | |
| Adnectin1_ | CTTTAAGAAGGAGAT | SEQ ID NO: 6 |
| for | ATACCATGGGGGTAT | |
| | CTGACGTG | |
| Adnectin1_ | CAGGCTGCCGCCGGA | SEQ ID NO: 7 |
| rev | TCCGCCTTGCGAAGG | |
| | TTTGTCGATTTC | |
| tFhuA_for | CCGGCGGATCCGGCG | SEQ ID NO: 8 |
| | GCAGCCTGAAAGAAG | |
| tFhuA_rev | GATCCTCGAGTTAAA | SEQ ID NO: 9 |
| | AACGAAAGGTTGCGG | |
| | TGGC | |
| Egfr_ecd_ | TCCGCTAGCGCCACC | SEQ ID NO: 10 |
| for | ATGGTGCGACCCTCC | |
| | GGGACG | |
| Egfr_ecd_ | TCGAGATCTTTAGTG | SEQ ID NO: 11 |
| rev | ATGATGATGATGATG | |
| | GGACGGGATCTTAGG | |
| | CCCATTCGTTGG | |
| tFhuA_cys_ | TGTGAAGGTAGCAGC | SEQ ID NO: 12 |
| for | GGTCCGTATCG | |
| tFhuA_cys_ | TGAATTATAGCCAAA | SEQ ID NO: 13 |
| rev | CCAGGCATTAATATC | |
| | ATTGCGC | |

Protein Expression and Purification

For the expression of FN3SUMO-tFhuA, Mb4-tFhuA, and Adnectin1-tFhuA, the plasmids mentioned above were transformed into E. coli BL21(DE3) cells. These monobody-containing protein nanopores were purified as previously described. The protein purity was validated by SDS-PAGE analysis (FIG. 2).

In the case of hSUMO1, BL21(DE3) cells were transformed with pET11a-hSUMO1 and grown in Luria-Bertani (LB) medium at 37° C. until OD₆₀₀ reached a value of ˜0.5. Then, the temperature was changed to 20° C. Expression was initiated by inducing the cells with 250 μM IPTG. After induction, the cells were cultured for ˜18 h at 20° C. Cells were then centrifuged at 3,700 g for 30 min at 4° C., followed by their resuspension in 50 mM Tris-HCl, 50 mM NaCl, and pH 8.0. The lysozyme was added to the suspended cells and incubated on ice for 15 min, and cell lysis was accomplished using sonication (30 s on, 60 s off×4 times). The cell lysate was centrifuged at 108,500 g for 30 min at 4° C. to separate the insoluble pellet and supernatant. The supernatant was collected and filtered using a 0.22 μm filter. The supernatant was loaded onto Q-Sepharose column (Cytiva, Marlborough, MA), which was washed with 50 mM Tris-HCl, 50 mM NaCl, pH 8.0, and eluted with 50 mM Tris-HCl, 1 M NaCl, pH 8.0 in a gradient manner. The desired fractions were collected, dialyzed, and concentrated. Furthermore, the protein sample was loaded on an S75 gel-filtration column (GE Healthcare, Chicago, IL). Pure fractions were collected and dialyzed against 20 mM Tris-HCl, 150 mM NaCl, pH 8.0, and 0.5 mM TCEP overnight at 4° C. Purification of WDR5 was done as described previously.

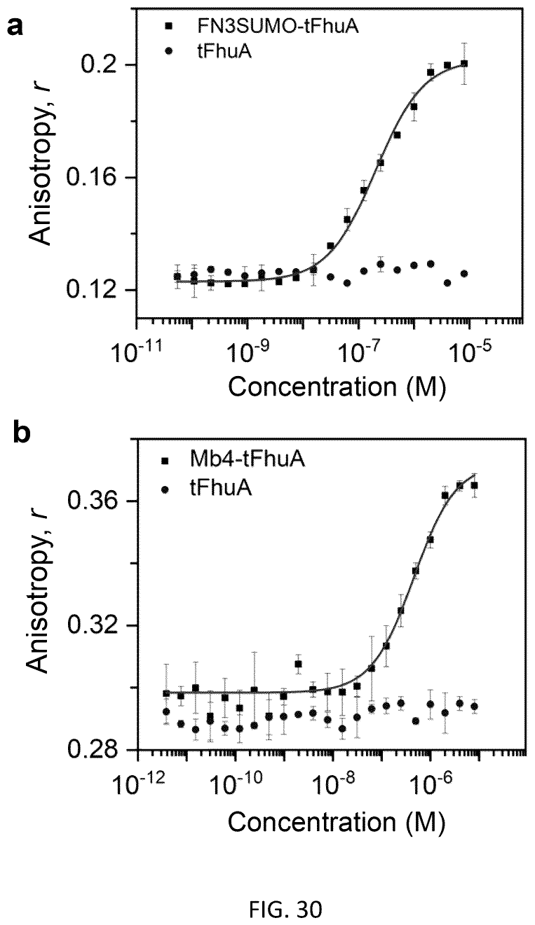

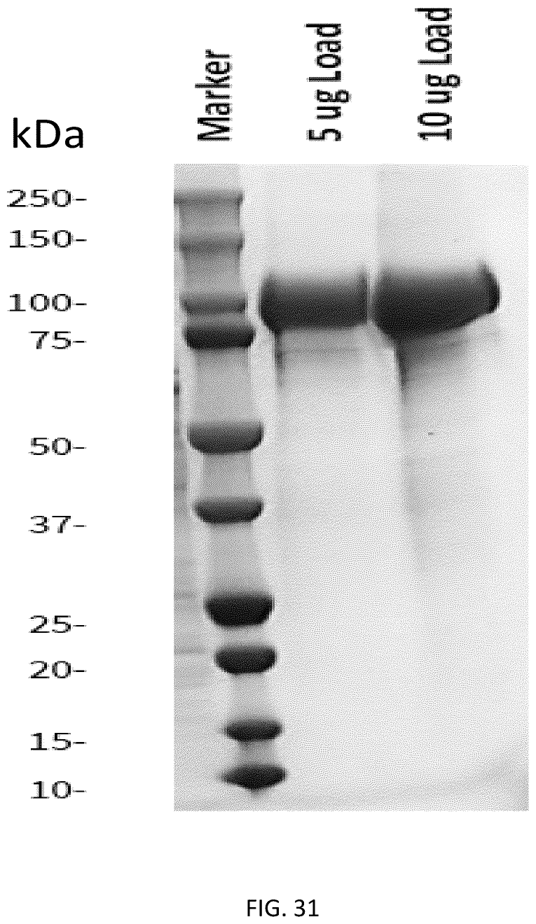

For the purification of the ectodomain of epidermal growth factor receptor (EGFR), Expi293F cells (Thermo Fischer Scientific) were seeded at 10⁶ cells/ml density in 1 L of Dynamis growth medium (Gibco) 24 h before the transfection and supplemented with Tryptone/Glucose. For the sake of simplicity, we name this EGFR throughout this article. The culture was transfected with 2 μg/mL of the pCMV_EGFR plasmid containing the signal peptide with 3.75×polyethylenimine (PEI). Transfected cells were cultured for five days, and the protein was allowed to excrete from the cells. Five days post-transfection, the culture was pelleted, and the supernatant was filtered. The sample was loaded onto an immobilized metal-affinity column (1 mL, HIStrap HP column, GE Healthcare), which was washed with 50 mM sodium phosphate (NaPi) (pH 8.0), 300 mM NaCl, 20 mM imidazole. The protein was eluted using 50 mM NaPi (pH 8.0), 300 mM NaCl, and 500 mM imidazole. Peak fractions were collected and confirmed by SDS-PAGE (FIG. 30). Finally, the protein sample was concentrated and buffer exchanged with phosphate buffer saline (PBS, pH 7.5) using a PD10 column (GE Healthcare) and stored at −80° C. The purity of all protein analytes was tested by SDS-PAGE analysis.

Protein Refolding

The purified FN3SUMO-tFhuA, Mb4-tFhuA, and Adnectin1-tFhuA were adjusted to a final concentration of ˜10 μM. Next, n-dodecyl-β-d-maltopyranoside (DDM) was added to denatured samples to a final concentration of 1% (w/v). The protein samples were immediately dialyzed against the buffer containing 200 mM KCl, 20 mM Tris-HCl, pH 8, at 4° C. for 96 h. The dialysis solution was replaced at 24-h intervals. These refolded protein samples were centrifuged to eliminate any protein precipitations, and the supernatant was used as the running sample for single-channel electrical recordings. Protein concentrations were determined by their molar absorptivity at a wavelength of 280 nm.

Single-Channel Electrical Recordings

Electrical detection of protein ligands at single-molecule precision was conducted using planar lipid bilayers. The two halves of the chamber were divided by a 25 μm-thick Teflon septum (Goodfellow Corporation, Malvern, PA). A planar lipid bilayer was made of 1,2-diphytanoyl-sn-glycero-phosphatidylcholine (Avanti Polar Lipids, Alabaster, AL) across an ˜100 μm-diameter aperture of the Teflon septum. For all experiments, the buffer solution contained 300 mM KCl, 10 mM Tris-HCl, and pH 8.0. In addition, this buffer included 0, 0.5, and 1 mM TCEP in experiments with EGFR, hSUMO1, and WDR5, respectively. The nanopore protein samples (final concentration, 0.5-1.5 ng/μ1) and analytes were added to the cis compartment, which was grounded. Single-channel electrical currents were acquired using an Axopatch 200B patch-clamp amplifier (Axon Instruments, Foster City, CA). The applied transmembrane potential was +40 mV, unless otherwise stated. The electrical signal was sampled at 50 kHz using a low-noise acquisition system (Model Digidata 1440 A; Axon Instruments). A low-pass Bessel filter (Model 900; Frequency Devices, Ottawa, IL) was further employed for signal filtering at 10 kHz. For the data processing and analysis, the electrical traces were digitally filtered with a low-pass 8-pole Bessel filter at 3 kHz, unless otherwise stated. All single-channel electrical recordings were acquired at a temperature of 24±1° C.

EGFR Detection in a Heterogeneous Solution

Fetal bovine serum (FBS, Gibco, Thermo Fisher Scientific, Pittsburgh, PA) was sterile-filtered using a 0.2-μm filter and stored at −80° C. in aliquots for long-term use. A fresh aliquot was defrosted on ice and incubated for at least 30 minutes at room temperature before adding to the chamber. Single-channel electrical traces were recorded in the presence of FBS at a final concentration of 5%.

Biolayer Interferometry (BLI) Assay Using Immobilized Proteomicelles

These experiments were conducted using an Octet Red384 instrument (FortéBio, Fremont, CA) at 24° C. For BLI experiments, a site specific insertion of cysteine at position 287 was achieved in the long loop of Mb4-tFhuA by site directed mutagenesis (Q5 mutagenesis kit, New England Biolabs). This cysteine-containing Mb4-tFhuA was expressed and purified as described above except for the presence of reducing agent. Cys287 was biotinylated using maleimide. A flexible (PEG)₁₁ linker was used between the biotin and maleimide group. In this way, there was a satisfactory distance between Mb4 and the surface of BLI sensor. The BLI running buffer contained 300 mM KCl, 20 mM Tris-HCl, 1 mM TCEP, 1% DDM, 1 mg/ml bovine serum albumin (BSA), pH 8.0. It was used to soak streptavidin (SA) sensors for 30 min. The 50 nM Mb4-tFhuA_Cys287-(PEG)₁₁-Biotinyl was loaded onto the sensors for 2.5 min via biotin-streptavidin chemistry. By dipping the sensors in a protein-free solution for 6 minutes, the unattached Mb4-tFhuA_Cys287 was washed away. The association process was examined using various concentrations of WDR5, ranging from 1.5 μM to 6 μM. To inspect the dissociation phase, the BLI sensors were dipped in a WDR5-free running buffer. For all WDR5 concentrations, the Mb4-tFhuA_Cys287-free BLI sensors were run in parallel as controls. The baseline and drift in the sensorgrams were subtracted using these controls. The FortéBio Octet data analysis software (ForteBio) was used for the sensorgram analysis.

Steady-State Fluorescence Polarization (FP) Measurements

hSUMO1 and WDR5 were labeled with fluorescein and rhodamine, respectively, at pH9.0 by primary amine chemistry. These labeled proteins were added to the well at a final concentration of 50 nM. Steady-state fluorescence polarization (FP) anisotropy assays were conducted in triplicate with an 18-point serial dilution of FN3SUMO-tFhuA, Mb4-tFhuA, or unmodified tFhuA, against a fixed concentration of labeled proteins on black 96-well plates. All steady-state FP measurements were recorded using a SpectraMax i3x plate reader (Molecular Devices, San Jose, CA) at 0 min and after a one-hour incubation at room temperature in the dark. The resulting dose-response data were averaged and fitted using a logistic regression to obtain the dissociation constant (K_{D}) for each interaction.

Statistical Analysis

pClamp 10.7 (Axon Instruments) was used for the data acquisition and analysis. Capture and release events were collected using the single-channel event search in ClampFit 10.7 (Axon Instruments), and figures were prepared by Origin 9.7 (OriginLab, Northampton, MA). The probability distribution function (PDF) was generated using a kinetic rate matrix, and the kinetic rates were determined by fitting the data using the maximum likelihood method. To evaluate the results of multiple models and select the number of statistically significant peaks that are best matched to the data, a logarithm likelihood ratio (LLR) test was performed. At a confidence number of 0.95, a single-exponential fit was the best model for the release and capture durations of hSUMO1 and WDR5. For EGFR, a two-exponential fit was the best model for the capture durations.

Molecular Graphics

All cartoons showing molecular graphics were prepared using the PyMOL 2 (Version 2.4.0; Schrödinger, LLC) and Chimera X (Version 1.4; University of California at San Francisco).

### Example 2

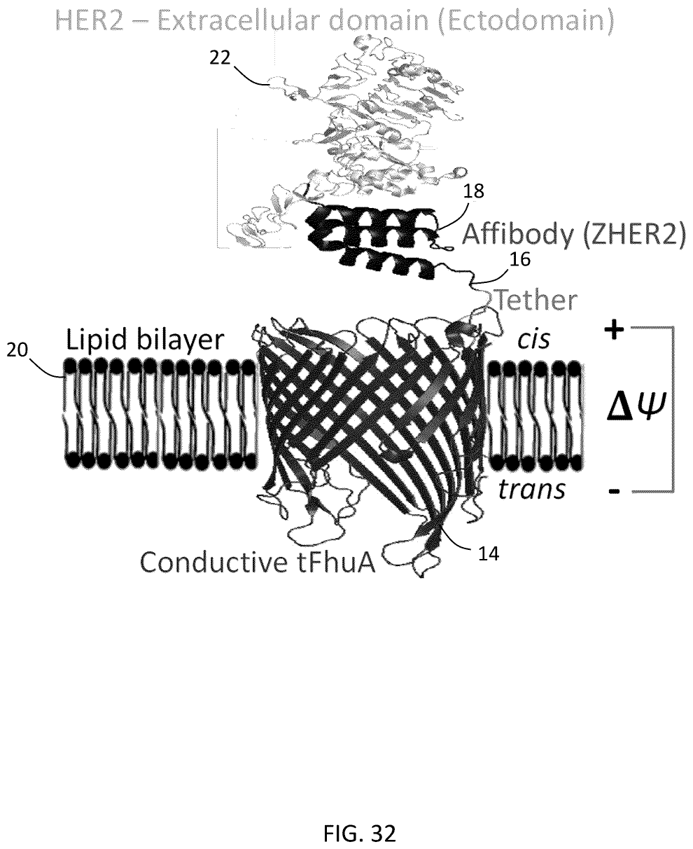

In another example of sensor 10 as seen in FIG. 32, affibodies were tested and confirmed as acceptable antibody mimetic scaffold and binding sites. Affibodies comprising 6.5-kDa proteins based on the Z domain of staphylococcal protein A and having a 58-residue three-helix bundle protein that is the critical domain binding to IgG were incorporated into sensor 10. Affibodies are advantageous due to their small size, convex shape, high thermostability, and absence of disulfide bonds. Moreover, high-affinity affibodies (e.g., with a K_{D} in the low pM) for the ectodomains of all human epidermal growth factor receptors (HERs) can, when incorporated into pore-based protein sensor 10, detect analytes several orders of magnitude below the K_{D}, i.e., an affibody-based sensor will exhibit an LOD of protein biomarkers in the femtomolar to the attomolar range. Human epidermal growth factor receptor 2 (HER2), a protein biomarker overexpressed in several cancers, may be bound using an affibody referred to as ZHER2, which exhibits a K_{D} of 22 pM. HER2 is of interest for detection in serum because the ectodomain of HER2 is released in the bloodstream through a proteolytic process, also called ectodomain shedding. Under physiological conditions, the threshold for elevated HER2 ectodomain level is 15 ng/ml (˜37 nM), according to FDA recommendations, whereas a change of 20% above this level is considered significant. Other relevant binding sites for use with affibodies include tumor necrosis factor α (TNF-α), interleukins IL-6 and IL-17, the ectodomains of epidermal growth factors receptors EGFR and HER3, the ectodomains of vascular endothelial growth factors receptors VEGFR-1 and VEGFR-2, and insulin-like growth factor-1 receptor (IGF-1R). In addition to affibodies, nanobodies which are recombinantly produced antigen binding V_{H}H fragments, may additionally be tethered to pore 14.

What is claimed is:

1. A sensor, comprising:
a lipid membrane;
a protein pore embedded in the membrane; and
a binding scaffold coupled to the protein pore by a tether and including an antibody-mimetic binder having a binding affinity for a target analyte;
wherein the lipid membrane has a first membrane potential when the target analyte is not bound to the antibody-mimetic binder and a second, different membrane potential when the target analyte is bound to the antibody-mimetic binder.

2. The sensor of claim 1, wherein the protein pore comprises a monomeric β-barrel scaffold.

3. The sensor of claim 2, wherein the monomeric β-barrel scaffold is a monomeric β-barrel scaffold of a tFhuA protein.

4. The sensor of claim 1, wherein the binding scaffold is an FN3 monobody.

5. The sensor of claim 1, wherein the tether comprises a (GGS)₂ tether.

6. The sensor of claim 5, wherein the (GGS)₂ tether is coupled to an N-terminus of the monomeric β-barrel scaffold of the tFhuA protein.

7. The sensor of claim 1, wherein the binding scaffold is an affibody.

8. The sensor of claim 1, wherein the binding scaffold is a peptide having less than 150 residues.

9. The sensor of claim 1, wherein the protein pore, tether, and antibody-mimetic binder is a single polypeptide having the sequence of SEQ ID NO: 1.

10. A method of detecting a target analyte, comprising the steps of:
providing a lipid membrane, a protein pore embedded in the membrane, and a binding scaffold coupled to the protein pore by a tether and including an antibody-mimetic binder having a binding affinity for the target analyte, wherein the lipid membrane has a first membrane potential when the target analyte is not bound to the antibody-mimetic binder and a second, different membrane potential when the target analyte is bound to the antibody-mimetic binder;
exposing the sensor to a solution that potentially contains the target analyte; and
detecting whether the first membrane potential has changed to the second membrane potential.

11. The method of claim 10, wherein the protein pore comprises a monomeric β-barrel scaffold.

12. The method of claim 11, wherein the monomeric β-barrel scaffold is a monomeric β-barrel scaffold of a tFhuA protein.

13. The method of claim 10, wherein the binding scaffold is an FN3 monobody.

14. The method of claim 10, wherein the tether comprises a (GGS)₂ tether.

15. The method of claim 14, wherein the (GGS)₂ tether is coupled to an N-terminus of the monomeric β-barrel scaffold of the tFhuA protein.

16. The method of claim 10, wherein the binding scaffold is an affibody.

17. The method of claim 10, wherein the binding scaffold is a peptide having less than 150 residues.

18. The method of claim 10, wherein the protein pore, tether, and antibody-mimetic binder is a single polypeptide having the sequence of SEQ ID NO: 1.
